(19) United States
(12) Patent Application Publication
(10) Pub. No.: US 2005/0053667 A1
(43) Pub. Date: Mar. 10, 2005

(54) Programmed immune responses using a vaccination node

(76) Inventors: Darrell Irvine, Arlington, MA (US); Siddhartha Jain, New Delhi (IN); Nir Hacohen, Cambridge, MA (US); William Warren, Orlando, FL (US)

Correspondence Address: Supervisor, Patent Prosecution Services;PIPER RUDNICK LLP, 1200 Nineteenth Street, N.W., Washington, DC, 20036-2412, US

(21) Appl. No.: 10/886,707

(22) Filed: Jul. 9, 2004

Related U.S. Application Data
(60) Provisional application No. 60/485,803, filed on Jul. 9, 2003.
(60) Provisional application No. 60/569,618, filed on May 11, 2004.

(51) Int. Cl. A61K 39/38; A61K 39/12; A61K 9/16; A61K 9/50

(52) U.S. Cl. 424/490; 424/204.1; 424/234.1

(57) ABSTRACT

The present invention provides compositions and methods for modulating immune responses to antigens. One aspect of the present invention relates to a particle-based antigen delivery system (vaccination node) that comprises a hydrogel particle capable of both antigen presentation and DC activation. The VN may further comprise a chemoattractant-loaded microsphere capable of attracting DCs to the site of administration. Another aspect of the present invention relates to the use of the VN to modulate antigen presenting cells activation for the prevention and/treatment of various diseases, such as infectious diseases, cancers and autoimmune diseases.

This application claims priority from U.S. Provisional Application Ser. No. 60/485,803, filed Jul. 9, 2003 and U.S. Provisional Application Ser. No. 60/569,618, filed May 11, 2004, respectively. The entirety of both provisional applications is incorporated herein by reference.

### FIELD OF THE INVENTION

The present invention relates to the field of immunotherapy and vaccine development. More particularly, it relates to a particle-based subunit vaccine that mimics immunological cascade of events to destroy invading pathogens. The particle-based vaccine is especially useful in modulating immunological responses against various diseases, such as autoimmune diseases, infectious diseases and cancers.

### BACKGROUND OF THE INVENTION

Vaccination with protein antigens (e.g., a virus protein or a tumor-specific antigen) is a new strategy that has tremendous clinical potential because of its low toxicity and widespread applicability. However, protein-based vaccines have had only limited clinical success because of the following reasons.

First, protein-based vaccines have delivery problems. Specifically, the effective utilization of protein therapeutics require the development of materials that can deliver bioactive material to diseased tissues and cells. At present, the majority of protein delivery vehicles are based on hydrophobic polymers, such as poly(lactide-co-glycolide) (PLGA). See O'Hagan, D. et al., in U.S. Pat. Nos. 6,306,405 and 6,086,901, and in Adv. Drug Delivery Rev, 32, 225 (1998). However, PLGA based delivery vehicles have been problematic because of their poor water solubility. Proteins are encapsulated into PLGA based materials through an emulsion procedure that exposes them to organic solvents, high shear stress and/or ultrasonic cavitation. This procedure frequently causes protein denaturation and inactivation. [Xing D et al., Vaccine, 14:205-213 (1996)].

Hydrogels have been proposed as an alternative protein delivery vehicle because they can encapsulate the protein in a totally aqueous environment under mild conditions. [See Park, K. et al., Biodegradable Hydrogels for Drug Delivery; Technomic Publishing Co, Lancaster, Pa. (1993); Peppas. N. A., Hydrogels in Medicine and Pharmacy; CRC Press: Vol II, Boca Raton, Fla., (1986); and Lee, K. Y. et al., Chemical Reviews, 101:1869-1179 (2001)]. Hydrogel is a colloidal gel in which water is the dispersion medium. Micron sized protein loaded hydrogel particles are small enough to be phagocytosed. At present, micron sized hydrogels have been synthesized using crosslinkers that do not degrade under biological conditions, and hence have had limited success in drug delivery applications.

Several advantages make hydrogel technology attractive for the intracellular and extracellular drug delivery applications described above. The encapsulation approach is applicable to several types of biopolymers of interest for vaccination and immunotherapy: purified proteins, peptides, DNA (for genetic immunization), polysaccharides, and whole cell lysates (of interest for immunization against tumors or poorly defined allergens). The ligand-modifiable gel particles encapsulate extremely large weight fractions of antigen (˜75 wt % of particles is encapsulated biopolymer in the example below). This is in contrast to approaches such as polyester microspheres, where maximal loading is typically less than 30 wt % and often less than 10 wt %. The stability of the gel particles is superior to liposomes, which are known to ‘leak’ entrapped drug rapidly and unpredictably. The gel particles retain encapsulated biopolymers with minimal loss for up to one week in suspension. Finally, the ability to tailor the breakdown of the particles by inclusion of peptide or synthetic polymer sequences sensitive to the local environment is a major advantage over other particulate drug delivery techniques.

Another limitation of protein-based vaccines is their inability to activate cytotoxic T lymphocytes (CTL). The activation of CTL is critical for the development of immunity against viruses and tumors. CTL is activated by dendritic cells (DCs) through the Class I antigen presentation pathway. DCs are derived from hematopoietic stem cells in the bone marrow and are widely distributed as immature cells within all tissues, particularly those that interface with the environment (e.g. skin, mucosal surfaces) and in lymphoid organs. Immature DCs are recruited to sites of inflammation in peripheral tissues following pathogen invasion. Internalization of foreign antigens can subsequently trigger their maturation and migration from peripheral tissues to lymphoid organs. Chemokine responsiveness and chemokine receptor expression are essential components of the DC recruitment process to sites of inflammation and migration to lymphoid organs. Following antigen acquisition and processing, DCs migrate to T cell-rich areas within lymphoid organs via blood or lymph, simultaneously undergoing maturation and modulation of chemokine and chemokine receptor expression profiles.

Immature DCs capture antigens by phagocytosis, macropinocytosis or via interaction with a variety of cell surface receptors and endocytosis. Following antigen processing, antigenic peptides may then be presented via MHC molecules on the DC surface to CD4⁺, CD8⁺ or memory T cells. DCs are capable of processing both endogenous and exogenous antigens and present peptide in the context of either MHC class I or II molecules. Typically, exogenous antigens are internalized, processed, and loaded onto MHC class II molecules; while endogenous antigens are loaded onto MHC class I molecules. For example, when DCs are themselves infected with a virus, proteasomes will degrade the viral proteins into peptides and transport them from the cytosol to the endoplasmic reticulum. A variety of cell surface receptors expressed by immature DCs may function in antigen uptake and also present antigen via the MHC I pathway.

Following antigen exposure and activation, DCs migrate into T cell areas of lymphoid organs, a process regulated by chemokine/chemokine receptor interaction and aided by a variety of proteases and corresponding receptors. Cell surface receptors not only facilitate antigen uptake, but also mediate physical contact between DCs and T cells. The soluble cytokine profile secreted by DCs varies with the different stages of DC development and maturation, and influences the different effector functions characteristic of immature and mature DCs. A wide variety of cytokines may be expressed (not necessarily simultaneously) by mature DCs including IL-12, IL-1a, IL-1b, IL-15, IL-18, IFNα, IFNβ, IFNγ, IL-4, IL-10, IL-6, IL-17, IL-16, TNFα, and MIF. The exact cytokine repertoire expressed will depend on the nature of the stimulus, maturation stage of the DC and the existing cytokine microenvironment.

In summary, DCs are unique antigen-presenting cells (APCs) as they can both initiate and modulate immune responses. Even small numbers of DCs and low levels of antigen can elicit strong immune responses. Therefore, manipulation of DC activation and maturation may translate into effective therapeutic interventions.

### SUMMARY OF THE INVENTION

The present invention provides compositions and methods for modulating immune responses to antigens, including foreign and self antigens.

One aspect of the present invention is directed to a particle-based antigen delivery system that comprises a hydrogel particle capable of both antigen presentation and DC activation. The hydrogel particle comprises an immunogen encapsulated in the hydrogel particle and a ligand on a surface of the hydrogel particle. The surface ligand interacts with an antigen presenting cell (e.g. a dendritic cell, a precursor of dendritic cell, a monocytes or a macrophage) and providing an activation or a maturation signal or both to the antigen presenting cell. The particle-based antigen delivery system is hereinafter referred to as a vaccination node (VN). The VN may further comprise a chemoattractant-loaded microsphere capable of attracting immature DCs and DC precursors to the site of administration.

Another aspect of the present invention is directed to the use of the VN to modulate DC activation and maturation. In one embodiment, the VN is used to stimulate immune responses to an antigen in order to eliminate a pathogen or a cancerous cell. In another embodiment, the VN is used to suppress immune responses for the treatment of autoimmune diseases or allergic reactions.

Yet another aspect of present invention relates to methods for making the hydrogel particles of the present invention and methods for forming micro-colloidal micelle VN particles comprising both the hydrogel particles and the chemoattractant-loaded microsphere.

### BRIEF DESCRIPTION OF THE FIGURES

FIG. 1 is a schematic of the salt-out hydrogel particle synthesis process.

FIGS. 2A and 2B illustrate multi-drug delivery platform utilizing ligand-modified biopolymer delivery hydrogel particles in vivo. FIG. 2A is a digitally-printed drug delivery device, multi-chamber depot. FIG. 2B shows the structure of the cross-section.

FIGS. 3A and 3B. FIG. 3A is the model for the life cycle of a dendritic cell in response to acute infections. FIG. 3B is a schematic of colloidal micelle vaccine system.

FIGS. 4A and 4B illustrate controlled release of MIP-3α from PLGA microspheres. FIG. 4A is the release profile which shows chemokine released into medium from microspheres over one week and detected by ELISA. FIG. 4B is the release rate calculated from the release profile as shown in FIG. 4A.

FIGS. 5A and 5B show the migration of dendritic cells in response to MIP-3α microspheres. Both figures illustrate two-dimensional plots of path endpoints. Arrows denotes direction toward microsphere source. FIG. 5A is the response to control ‘empty’ microspheres. FIG. 5B is the response to MIP-3α-releasing microspheres.

FIGS. 6A, 6B, 6C, 6D, and 6E show the controlled release of microspheres attract lymphocytes in vivo.

FIGS. 7A and 7B illustrate the characterization of antigen-delivery hydrogel particles. FIG. 7A shows photon correlation spectroscopy particle sizing data. FIG. 7B shows encapsulated TR-Ova fluorescence. False-Color fluorescence micrograph of Texas red-conjugated ovalbumin-loaded particles dried on a glass coverslip.

FIGS. 8A, 8B and 8C illustrate the antigen delivery to dendritic cells in vitro. FIG. 8A shows the time-lapse fluorescence imaging of particle uptake by a DC. FIG. 8B is the flow cytometry analysis of propidium iodide stained dendritic cells incubated 24 hrs with antigen-loaded particles and controls incubated with media alone. FIG. 8C shows the activation of CD8⁺ T cells by particle-treated dendritic cells.

FIG. 9 illustrates the proposed mechanism of antigen processing of ova gel particles by dendritic cells. (1) is the particles taken up by endocytosis/phagocytosis; (2) is the low molar mass proteases diffuse into particles, and proteolyze the entrapped antigen, and (3) is the antigen fragments diffuse out of the particles to be processed by normal intracellular antigen processing pathways.

FIGS. 10A and 10B illustrate antigen release from ova gel particles by action of intracellular proteases. FIG. 10A shows the content of protein remaining in the particles after ova-loaded antigen delivery gel particles incubated with varying doses of cathepsin D in pH 5.5 buffer mimicking conditions within endosomes. FIG. 10B is time-course of protein release from particles exposed in vitro to the endosomal protease cathepsin D.

FIGS. 11A and 11B illustrate the activation and maturation of dendritic cells by CpG-antigen particles. FIG. 11A shows IL-12 production by immature bone marrow-derived dendritic cells triggered by incubation 24 hrs with particles, soluble CpG, or CpG-modified particles. FIG. 11B is the flow cytometry analysis of MHC II (I-Ab) and CD86 expression by BMDCs in response to incubation 24 hrs with soluble CpG or equimolar levels of CpG bound to gel particles and the comparison of response to LPS.

FIGS. 12A, 12B and 12C illustrate the T cell activation by ova- or encapsulated ova-pulsed dendritic cells in vitro. FIG. 12A shows the IL-2 production by CD4⁺ OT-II T cell blasts after 24 hrs incubation with bone marrow-derived DCs pulsed with different concentrations of soluble ova or gel particle ova. FIG. 12B shows the IFN-γ production by OT-II T cell blasts after 24 hrs. FIG. 12C shows the IL-2 production by CD8⁺ OT-I T cells in response to BMDCs pulsed with varying concentration of soluble ovalbumin, ova particles, or control BSA particles.

FIGS. 13A, 13B and 13C illustrate the activation of naive T cells in vitro by particle-pulsed dendritic cells. FIG. 13A shows the proliferation of CD4⁺ OT-II naive T cells in response to different forms of ova antigen with or without CpG. FIG. 13B shows the percentages of cells dividing under each experimental condition determined from flow cytometry data. FIG. 13C shows the activation of native CD8⁺ OT-I cells-percentages of cells dividing after 60 hours as determined by flow cytometry.

FIGS. 14A and 14B illustrate the activation of naive CD4⁺ and CD8⁺ T cells by immunization with hydrogel antigen delivery particles in vivo. FIG. 14A shows the identification of the CFSE dilution from OT-II. FIG. 14B shows the identification of the CFSE dilution from OT-I.

FIGS. 15A and 15B illustrate the maturation pathway to be tested for optimal programming of dendritic cells in vivo.

### DETAILED DESCRIPTION OF THE INVENTION

The present invention relates to compositions and methods for modulating immune responses to antigens, including foreign and self antigens, using vaccination node (VN). In both immunization and natural infection-driven immune responses, antigen presenting cells (APCs), such as dendritic cells (DCs), play a critical role in initiating T cell activation, as they are the only cells known to have the capacity to prime naive T cells in vivo [Banchereau, et al., Nature, 392:245-52 (1998); Banchereau, et al., Annu Rev. Immunol., 18:767-811 (2000); and Norbury et al., Nat Immunol., 3:265-71 (2002)]. The VN is a particle-based vaccine composition that is capable of ‘engineering’ the local microenvironment at a vaccination site to program APCs, such as DCs, using controlled substance release and delivery technologies described herein. In modulating immune responses, the VN first acts like a hub to attract a wide array of various immune cells, such as neutrophils, monocytes, NK cells, macrophages, DCs (both mature and/or immature DCs), etc; and particularly, the VN may attract monocytes and/or immature DCs. Using micro and nano encapsulation particles, the VN creates an environment with the maturation proteins and DC modulators which allow the loaded DCs to become cross-primed, matured, and then subsequently, migrate to the patient's draining host lymph node (HLN).

In order to provide a clear and consistent understanding of the specification and claims, including the scope given to such claims, the following definitions are provided:

The term “biopolymer” refers to macromolecules that are involved in the structure or regulation of life processes. Examples of biopolymers include, but are not limited to, proteins, polypeptides, polynucleotides, polysaccharides, steroids, lipids, and mixtures thereof such as cell lysate.

The term “cell membrane protein,” as used herein, is any protein associated with a cellular membrane, including proteins having an extracellular domain and proteins situated on the surface, or in the lipid bi-layer, of the cell membrane. The proteins may be glycoproteins. Preferably, the proteins are surface antigens of a tumor cell. The cellular membrane may be that of a single cell, such as from a multicellular organism, more preferably a mammalian cell, and most preferably a tumor cell.

An “immune response” to an antigen is the development in a mammalian subject of a humoral and/or a cellular immune response to the antigen of interest. A “cellular immune response” is one mediated by T lymphocytes and/or other white blood cells. One important aspect of cellular immunity involves an antigen-specific response by cytotoxic T lymphocytes (“CTL”s). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes.

The term “antigen” as used herein, refers to any agent (e.g., any substance, compound, molecule [including macromolecules], or other moiety), that is recognized by an antibody, while the term “immunogen” refers to any agent (e.g., any substance, compound, molecule [including macromolecules], or other moiety) that can elicit an immunological response in an individual. These terms may be used to refer to an individual macromolecule or to a homogeneous or heterogeneous population of antigenic macromolecules. It is intended that the term encompasses protein molecules or at least one portion of a protein molecule, which contains one or more epitopes. In many cases, antigens are also immunogens, thus the term “antigen” is often used interchangeably with the term “immunogen.” The substance may then be used as an antigen in an assay to detect the presence of appropriate antibodies in the serum of the immunized animal.

The term “non-self antigens” are those antigens or substances entering a mammal, or exist in a mammal but are detectably different or foreign from the mammal's own constituents, whereas “self” antigens are those which, in the healthy subject, are not detectably different or foreign from its own constituents. However, under certain conditions, including in certain disease states, an individual's immune system will identify “non-self” antigens as its own constituents as “self,” and will not initiate an immune response against “non-self”. Conversely, an individual's immune system may also identify “self” antigens as “non-self,” and mount an immune response against the “self” antigens, leading to auto-immune diseases. The “self” antigen may also be used as an immunogen to induce tolerance in the treatment of autoimmune diseases.

“Tumor-specific antigen(s)” refers to antigens that are present only in a tumor cell at the time of tumor development in a mammal. For example, a melanoma-specific antigen is an antigen that is expressed only in melanoma cells but not in normal melanocytes.

“Tissue-specific antigen(s)” refers to antigens that are present only in certain kinds of tissues at a certain time in a mammal. For example, a melanocyte-specific antigen is an antigen that is expressed in all melanocytes, including normal melanocytes and abnormal melanocytes.

“Tissue graft antigen(s)” refers to antigens involved in graft-verses-host diseases. Tissue graft antigen determines acceptance or rejection of a tissue graft by the immune system. Examples of tissue graft antigens include, but are not limited to, histocompatibility antigens.

The term “monovalent” refers to a vaccine which is capable of provoking an immune response in a host animal directed against a single type of antigen. In contrast, a “multivalent” vaccine provokes an immune response in a host animal directed against several (i.e., more than one) toxins and/or enzymes associated with disease (e.g., glycoprotease and/or neuraminidase). It is not intended that the vaccine be limited to any particular organism or immunogen.

As used herein, the term “autoimmune disease” means a set of sustained organ-specific or systemic clinical symptoms and signs associated with altered immune homeostasis that is manifested by qualitative and/or quantitative defects of expressed autoimmune repertoires. Autoimmune diseases are characterized by antibody or cytotoxic immune responses to epitopes on self antigens found in the diseased individual. The immune system of the individual then activates an inflammatory cascade aimed at cells and tissues presenting those specific self antigens. The destruction of the antigen, tissue, cell type, or organ attacked by the individual's own immune system gives rise to the symptoms of the disease. Clinically significant autoimmune diseases include, for example, rheumatoid arthritis, multiple sclerosis, juvenile-onset diabetes, systemic lupus erythematosus (SLE), autoimmune uveoretinitis, autoimmune vasculitis, bullous pemphigus, myasthenia gravis, autoimmune thyroiditis or Hashimoto's disease, Sjogren's syndrome, granulomatous orchitis, autoimmune oophoritis, Crohn's disease, sarcoidosis, rheumatic carditis, ankylosing spondylitis, Grave's disease, and autoimmune thrombocytopenic purpura.

The term “antigen desensitization” refers to the process of decreasing an immune response by delivering to a mammalian subject, over a period of time, the antigen against which an immune response is mounted. With repeated exposure of the immune cells to the antigen, a decrease in the cytotoxic response is seen. Such desensitization can include, but is not limited to, a switch from a TH 1-like response to a TH 2-like response to the subject antigen. Antigen desensitization can be used for the treatment of autoimmune and allergic diseases.

An “allergen” is an immunogen which can initiate a state of hypersensitivity, or which can provoke a hypersensitivity reaction in a mammalian subject already sensitized with the allergen. An allergen can be a biopolymer, an environmental immunogen (e.g. pollen), or a non-nature, synthetic antigen.

One aspect of the present invention relates to a VN that is capable of attracting and subsequently programming mammalian DCs in vivo for the treatment and/or prevention of a particular disease. The VN typically comprises antigen delivery/DC maturation particles that provide encapsulated immunogen while simultaneously delivering maturation/activation signals to DCs. The VN may further comprises degradable microspheres that provide steady, controlled release of encapsulated chemoattractants. The antigen delivery/DC maturation particles and the degradable microspheres can be co-administered or physically associated prior to administration.

The antigen delivery/DC maturation particles of the present invention simultaneously deliver maturation/activation signals to DCs in an in vivo setting, mimicking interactions of DCs with pathogens which simultaneously provide antigenic material and stimulate DC maturation pathways. The in vivo activation of DC's has both time and cost advantages relative to traditional DC based vaccines. For example, total treatment time and costs are reduced since it is no longer necessary to isolate DC's from patients, expand the DC population in vitro, incubate the expanded DCs with an antigen in vitro, and re-inject the DCs.

The antigen delivery/DC maturation particles of the present invention are formed by polymerizing hydrogel precursor monomers in the presence of a salted-out aqueous immunogen emulsion. Suitable gel monomers include such hydrophilic and amphiphilic vinyl monomers as poly(ethylene glycol) [PEG] methacrylates and acrylates, poly(acrylic acid), poly(methacrylic acid), 2-diethylaminoethylmethacrylate, 2-aminoethyl methacrylate, poly(ethylene glycol) dimethacrylates and acrylates, poly(2-hydroxyethyl methacrylate), methacrylated dextrans, acrylated dextrans, acrylamide/bisacrylamide, poly(ethylene glycol)-polyester acrylated/methacrylated block copolymers (e.g. acrylated PEG-poly(lactide-co-glycolide) [PLGA]-PEG or PLGA-PEG-PLGA) and the like. Specific biopolymer functional groups may also be incorporated in the gel particles by copolymerization with peptide-modified monomers (such as acrylated/methacrylate PEG-peptide-PEG or PEG-peptide) [Irvine et al., Biomacromol., 2:85-94 (2001); West et al., Macromolecules, 32:241-244 (1999)]. The hydrogel particles typically have an average diameter of 10-1000 nm, preferably 200-600 nm.

The immunogens are encapsulated in the hydrogel particles. Examples of immunogens include biopolymers such as polypeptides, lipids, and polysaccharides that may serve as non-self or self antigens, tumor-specific antigens, tissue-specific antigens, tissue graft antigens. The immunogen also include polynucleotides that encode protein antigens or serve as antigens themselves. The advantage of using polynucleotides, such as a DNA construct capable of expressing an antigen, is that they are relatively inexpensive and generally more stable than polypeptides and polysaccharides. In addition, a DNA expression construct has the potential benefit of ‘unlimited’ antigen delivery—since each DC successfully transduced with the DNA construct could produce the antigen constitutively—creating ‘antigen factories’ at the immunization site. The immunogen further include antigens not found in nature (synthetic antigens) but have therapeutic efficacy for an immune-related disorder.

In one embodiment, the immunogens are biopolymers obtained or originated from microbes, such as Actinobacillus actinomycetemcomitans; Bacille Calmette-Gurin; Blastomyces dermatitidis; Bordetella pertussis; Campylobacter consisus; Campylobacter recta; Candida albicans; Capnocytophaga sp.; Chlamydia trachomatis; Eikenella corrodens; Entamoeba histolitica; Enterococcus sp.; Escherichia coli; Eubacterium sp.; Haemophilus influenzae; Lactobacillus acidophilus; Leishmania sp.; Listeria monocytogenes; Mycobacterium vaccae; Neisseria gonorrhoeae; Neisseria meningitidis; Nocardia sp.; Pasteurella multocida; Plasmodium falciparum; Porphyromonas gingivalis; Prevotella intermedia; Pseudomonas aeruginosa; Rothia dentocarius; Salmonella typhi; Salmonella typhimurium; Serratia marcescens; Shigella dysenteriae; Streptococcus mutants; Streptococcus pneumoniae; Streptococcus pyogenes; Treponema denticola; Trypanosoma cruzi; Vibrio cholera; and Yersinia enterocolitica.

In another embodiment, the immunogens are biopolymers obtained or originated from viruses, such as influenza virus; parainfluenza virus; rhinovirus; hepatitis A virus; hepatitis B virus; hepatitis C virus; apthovirus; coxsackievirus; Rubella virus; rotavirus; Denque virus; yellow fever virus; Japanese encephalitis virus; infectious bronchitis virus; Porcine transmissible gastroenteric virus; respiratory syncytial virus; Human immunodeficiency virus (HIV); papillomavirus; Herpes simplex virus; varicellovirus; Cytomegalovirus; variolavirus; Vacciniavirus; suipoxvirus and coronavirus.

In another embodiment, the immunogens are biopolymers obtained or originated from a parasite, such as protozoa and helminth.

In another embodiment, the immunogens are biopolymers obtained or originated from tumor specific antigens or other pathogens.

In yet another embodiment, the immunogens are a mixture of biopolymers such as cell lysates.

In yet another embodiment, the immunogen comprises a tissue graft antigen, a self antigen, or an allergen, and is administered for the induction of immune tolerance or for the suppression of an immune response.

The antigen delivery/DC maturation particles of the present invention are capable of intracellular delivery of the biopolymers to cells once internalized by endocytosis, phagocytosis, or macropinocytosis. In one embodiment, the antigen delivery/DC maturation particles of the present invention further contain peptide sequences and/or DNA plasmids that permit the selective release of the encapsulated biopolymers upon delivery of the particles to intracellular compartments. Specific release of encapsulated biopolymers can be obtained by several mechanisms. Hydrogel particles formed with non-degradable cross-links around a protein or peptide antigen will release antigen once internalized by phagocytes (DCs or macrophages) into endosomes and exposed to low molar mass proteases that may diffuse into the particle, degrade the biopolymer, and allow diffusion of biopolymer fragments out of the particle. This simple route is of interest for delivery of a polypeptide or polysaccharide antigen, where biopolymer degradation is a natural step in the processing of antigen. For applications where cleavage of the delivered biopolymer is undesirable (e.g. DNA delivery), the particle can be designed to specifically degrade on entry into endosomes by incorporation of cross-links containing enzyme-sensitive peptides or environment-sensitive (e.g., pH-sensitive) synthetic polymer sequences. An example is the use of cathepsin-sensitive peptide sequences that will be cleaved by cathepsins present in endosomes within cells. These linkages will be stable until particles are internalized to endosomes/phagosomes and exposed to cathepsins that can rupture the particles by enzymatic cleavage of the target peptide substrates.

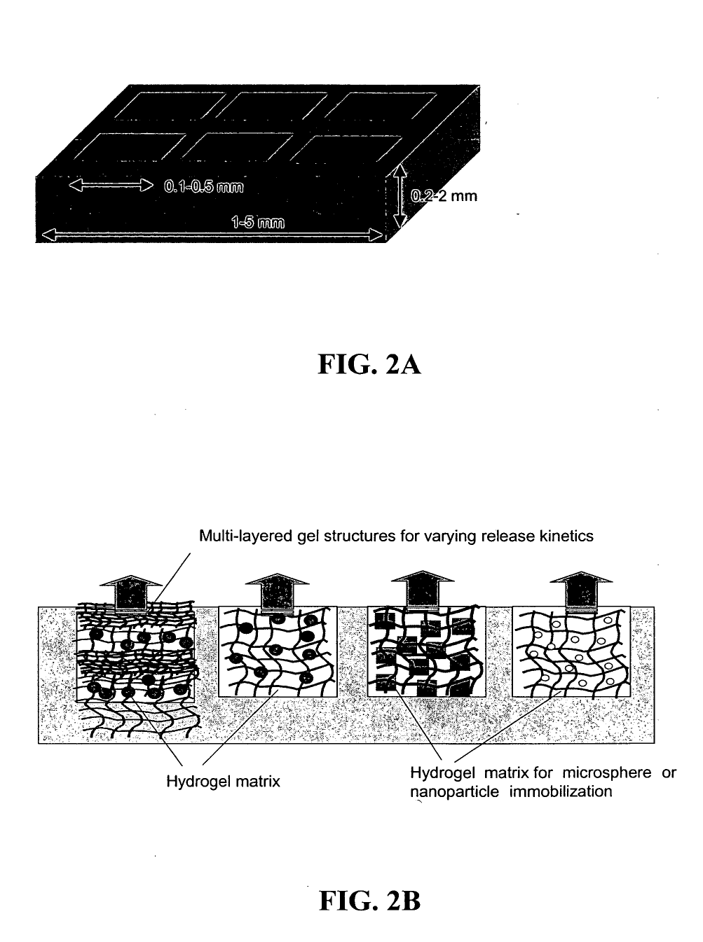

It is also conceivable that the antigen delivery/DC maturation particles can be used for gene therapy, general intracellular drug delivery, delivery of general sub-unit vaccines, delivery of anti-tumor compounds, or delivery of intracellular/cell surface signals for tissue engineering. These multi-signaling delivery particles may also be effective components of drug delivery devices, including platform-based devices such as illustrated in FIG. 2.

Moreover, The antigen delivery/DC maturation particles of the present invention are capable of encapsulating large weight fractions of antigen (˜75 wt % of particles is encapsulated biopolymer in the example below). This is in contrast to approaches such as polyester microspheres, where maximal loading is typically less than 30 wt % and often less than 10 wt % [Lavelle et al., Vaccine, 17:512-29 (1999); Jiang et al., Pharm Res., 18:878-85 (2001)]. The stability of the antigen delivery/DC maturation particles of the present invention is also superior to liposomes, and the antigen delivery/DC maturation particles of the present invention retain encapsulated biopolymers with minimal loss for up to one week in suspension. Finally, the ability to tailor the breakdown of the antigen delivery/DC maturation particles of the present invention by inclusion of peptide or synthetic polymer sequences sensitive to the local environment is a major advantage over other particulate drug delivery techniques.

In addition, the antigen delivery/DC maturation particles of the present invention can be used to deliver antigen to DCs as a vaccine, where antigen delivery to class I and class II loading pathways is desired, in addition to triggering activation of DCs via specific DC-surface receptors. A major difficulty in designing vaccines suitable for cancer or intracellular pathogens lies in obtaining CD8⁺ cytotoxic T cell (CTL) activation. CD8⁺ T cells are activated by foreign peptides presented on class I MHC molecules on the surface of DCs. DCs typically only load cytosolic peptides onto class I MHC, while exogenous antigens that are internalized are processed and loaded onto class II MHC molecules. Thus vaccines comprising free protein antigen do not elicit CTL responses, due to the lack of class I MHC loading of the antigen. However, it has recently been discovered that antigen delivered in a particulate form, either adsorbed to solid polymer microspheres [Raychaudhuri et al., Nat Biotechnol., 16:1025-31 (1998)], encapsulated in microspheres [Maloy et al., Immunology, 81:661-7 (1994)], or aggregated in the form of immunocomplexes with antibody [Rodriguez et al., Nat Cell Biol., 1:362-8 (1994)], triggers a ‘cross-presentation’ pathway that allows the antigen to be loaded on class I MHC. The antigen delivery/DC maturation particles of the present invention allow more efficient cross-presentation of the antigen to both MHC I and MHC II class molecules because of their ability to be loaded with large quantities of proteins without exposure to denaturing conditions.

In another embodiment, The antigen delivery/DC maturation particles of the present invention further contain ligands on their surface to target either cell surface receptors or components of extracellular matrix (ECM), thus facilitating the binding of the particles to cells or to specific sites in ECM. The ligands can be attached to the surface of the antigen delivery/DC maturation particles by covalent bonds or via non-covalently interactions, such as electrostatical interaction and streptavidin-biotin interaction.

The surface-modified particles allow simultaneous delivery of receptor-mediated signals or improve targeting of the particles to a specific cell type. Such particles allow the delivery of simultaneous signals both through the cell surface, via receptors binding the particle-surface ligand, and intracellularly, through biopolymers released from endocytosed particles. These particles may achieve two functions: (1) providing targeting of the particles to DCs, which specifically express receptors for the ligand (and if desired, other activation factors), and (2) triggering maturation of DCs once internalized in phagosomes, where they bind to the associated TLR receptors.

There are many ligands that are known to effect DC maturation/activation. It is thus possible to elicit a desired and tailored immune response by manipulating endpoint T cell activation via the attachment of different maturation signals to the surface of the particles. Examples of the particle surface ligands include, but are not limited to, CpG, CD40 ligand, vitamin D, dsRNA, poly(I:C), IL-2, IL-4, IL-7, IL-13, IL-15, LPS, bacterial lipoproteins, lipid A, TGF-β, TLR7 ligands (imidazoquinolines), antibodies against TLR receptors, and antibodies against DEC-205. The physical co-localization of antigen and maturation factors within the particles ensure that all DCs exposed to antigen are matured, and that only DCs receiving antigen receive maturation signals (to avoid autoimmune responses).

In another embodiment, the VN further comprises degradable, chemoattractants-loaded microspheres. The degradable microspheres provide steady, controlled release of encapsulated chemoattractants to attract various lymphocytes to migrate to a particular site.

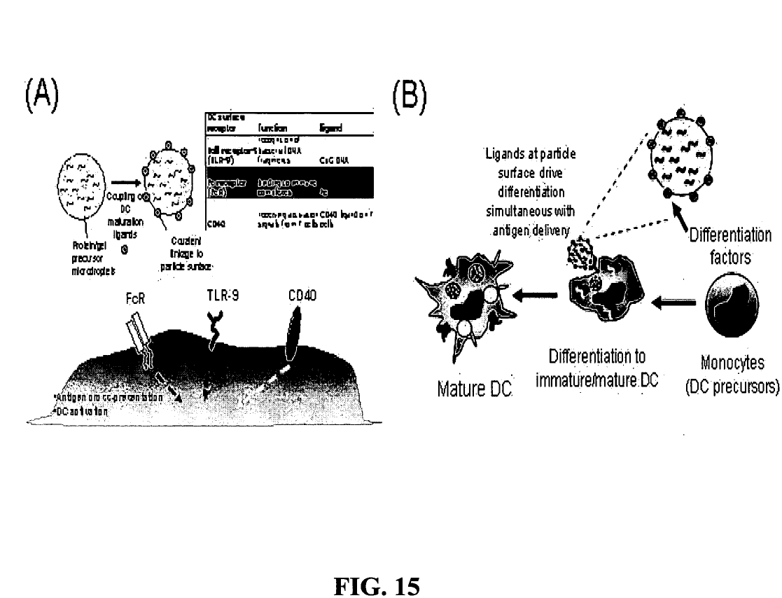

As illustrated in FIG. 15A, there are many known DC maturation/activation factors, all with different properties and effects on DC function. Different maturation signals manipulate endpoint T cell activation in vitro and in vivo to elicit a desired and tailored immune response. In particular, the effectiveness of CpG, antibody Fc and CD40 ligand, which bind to TLR-9, FcR and CD40 on the DC surface, respectively can all be designed in the VN, which combines antigen delivery/DC activation particles with the chemokine-releasing microspheres. The chemokine-releasing microspheres attract immature dendritic cells to an immunization site, where they can be efficiently primed and loaded with antigen by the antigen delivery/DC activation particles for T cell activation.

In another embodiment, the VN is also capable of releasing monocyte chemoattractants at the immunization site, and present to monocytes differentiation factors at the surface of the antigen delivery particles, as illustrated FIG. 15B. In this embodiment, the VN not only has the capability of attracting immature dendritic cells (which have a low prevalence in blood and tissues), but also dendritic cell precursors, such as monocytes, which might be differentiated by the VN in situ.

Degradable microspheres have been widely used in drug delivery systems. Examples of degradable microspheres include, but are not limited to, PEG and dextran block-copolymer particles having an average diameter of 1-500 um.

Examples of chemoattractants include, but are not limited to, cytokines such as IL-12, IL-1a, IL-1b, IL-15, IL-18, IFNα, IFNβ, IFNγ, IL-4, IL-10, IL-6, IL-17, IL-16, TNFα, and MIF; as well as chemokines such as MIP-3a, MIP-1a, MIP-1b, RANTES, MIP-3b, SLC, fMLP, IL-8, SDF-1α, and BLC.

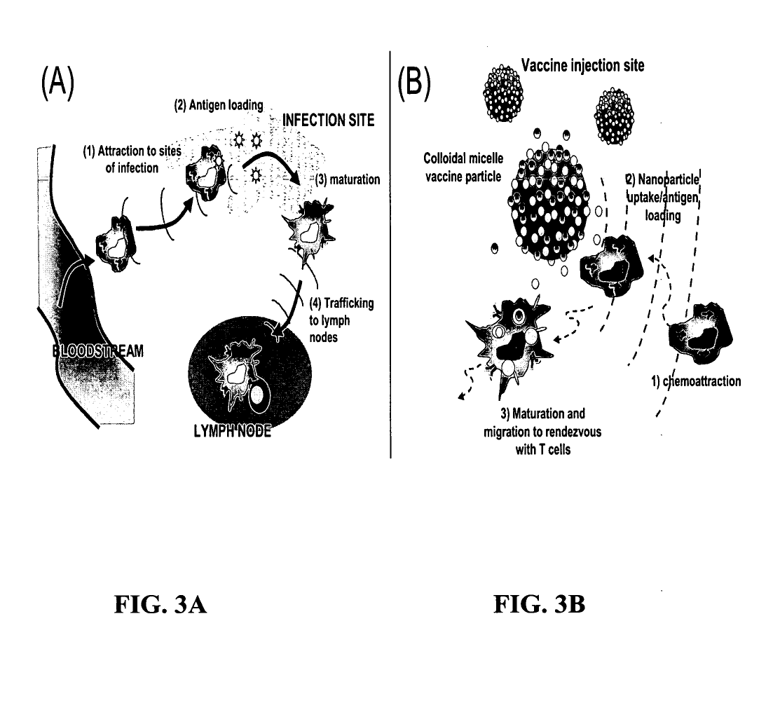

The assembly of colloidal micelles (illustrated in FIG. 3B) formed by binding antigen-delivery particles to the surface of chemokine-releasing microspheres, which will disassemble over time via degradation of the interparticle bonds has the following benefits: (1) These assembled super-particles will localize a high concentration of the antigen-delivery particles with each individual chemoattraction microsphere on injection of the colloidal micelle suspension, centering the antigen delivery/DC activation component at the chemoattractant source. (2) In addition, the delay in release into the local microenvironment will limit nonspecific removal of the particles by tissue macrophages and allow time for DC recruitment to the vaccine site.

The vaccine-chemotactic approach of VN eliminates delays normally associated with cell culturing and manipulation; reduces costs of having to maintain aseptic environments during manufacture, storage, shipment and delivery due to less rigorous standards applicable to non-organic materials; simplifies vaccine processing due to the absence of live cells; and allows faster FDA approval and lower developmental cost due to speedier, less stringent pre-clinical and clinical trial requirements.

Another aspect of the present invention relates to the synthesis of the VN. The antigen delivery/DC activation hydrogel particles are formed by polymerizing hydrogel precursor monomers in the presence of a salted-out aqueous protein, DNA, poly saccharide, or cellular lysate emulsion. Co-localization of the gel precursors in the protein-rich phase of the emulsion during polymerization leads to formation of gel particles whose sizes can be ˜0.01 μm to ˜50 μm, preferably, ˜0.05 μm to ˜50 μm, depending on the exact synthesis conditions. Polymerization can be initiated by standard free radical initiators, such as ammonium persulfate/sodium metabisulfite at 40° C. or by azobisisobutyronitrile at 60° C.

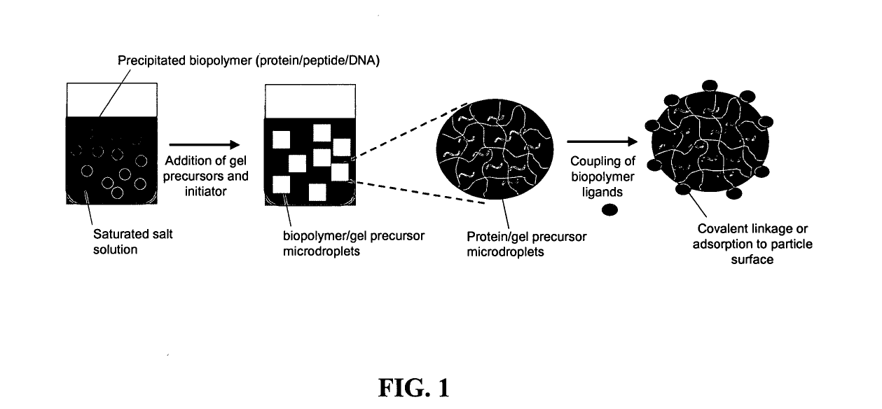

Inclusion of functional monomers in the synthesis that incorporate functional groups in the gel particles allows covalent attachment of other biopolymer ligands on the surface of the gel particles in a second step, to increase the functionality of the particles. A schematic of the particle synthesis process is presented in FIG. 1. The encapsulated biopolymer is retained by virtue of the high cross-link density within the gel particle network and/or specific interactions with functional groups within the network (such as electrostatic, hydrogen-bonding, or receptor-ligand interactions). Coupling of ligand to the surface of the particles may be covalent or non-covalent (e.g. through adsorption of protein to the particle surface).

Methods for making degradable microspheres and loading the microspheres with a controlled-release substance, such as a chemoattractant, can be found, for example, in U.S. Pat. Nos. 5,674,521; 5,980,948; and 6,303,148.

Yet another aspect of the present invention relates to methods for preventing or treating various diseases using the VN. Because the activation/maturation of DCs play an important rule in immune activation, the VN of the present invention may be used for the prevention or treatment of various diseases by activating the immune system. For example, the VN can be designed to target one disease at a time by controlling the maturation state of the DCs and/or loading them with the proper antigen. The VN can also be designed to provide an engineered environment for inducing tolerance.

In one embodiment, the VN of the present invention is administered into a mammal for the prevention or treatment of infectious diseases. Examples of infectious diseases include, but are not limited to, diseases caused by microbes such as Actinobacillus actinomycetemcomitans; Bacille Calmette-Gurin; Blastomyces dermatitidis; Bordetella pertussis; Campylobacter consisus; Campylobacter recta; Candida albicans; Capnocytophaga sp.; Chlamydia trachomatis; Eikenella corrodens; Entamoeba histolitica; Enterococcus sp.; Escherichia coli; Eubacterium sp.; Haemophilus influenzae; Lactobacillus acidophilus; Leishmania sp.; Listeria monocytogenes; Mycobacterium vaccae; Neisseria gonorrhoeae; Neisseria meningitidis; Nocardia sp.; Pasteurella multocida; Plasmodium falciparum; Porphyromonas gingivalis; Prevotella intermedia; Pseudomonas aeruginosa; Rothia dentocarius; Salmonella typhi; Salmonella typhimurium; Serratia marcescens; Shigella dysenteriae; Streptococcus mutants; Streptococcus pneumoniae; Streptococcus pyogenes; Treponema denticola; Trypanosoma cruzi; Vibrio cholera; and Yersinia enterocolitica. Further examples include diseases caused by viruses, such as influenza virus; parainfluenza virus; rhinovirus; hepatitis A virus; hepatitis B virus; hepatitis C virus; apthovirus; coxsackievirus; Rubella virus; rotavirus; Denque virus; yellow fever virus; Japanese encephalitis virus; infectious bronchitis virus; Porcine transmissible gastroenteric virus; respiratory syncytial virus; Human immunodeficiency virus (HIV); papillomavirus; Herpes simplex virus; varicellovirus; Cytomegalovirus; variolavirus; Vacciniavirus; suipoxvirus and coronavirus.

In another embodiment, the VN of the present invention is administered into a mammal for the prevention or treatment of cancer. Examples of cancer include, but are not limited to, breast cancer, colon-rectal cancer, lung cancer, prostate cancer, skin cancer, osteocarcinoma, and liver cancer.

Because the DCs naturally foster tolerance by the immune system, the VN of the present invention can be administered into a mammal for the treatment of autoimmune diseases. Examples of such diseases include, but are not limited to, asthma, systemic lupus erythematosus (SLE), rheumatoid arthritis, multiple sclerosis, juvenile-onset diabetes, autoimmune uveoretinitis, autoimmune vasculitis, bullous pemphigus, myasthenia gravis, autoimmune thyroiditis or Hashimoto's disease, Sjogren's syndrome, granulomatous orchitis, autoimmune oophoritis, Crohn's disease, sarcoidosis, rheumatic carditis, ankylosing spondylitis, Grave's disease, and autoimmune thrombocytopenic purpura.

In one aspect, the present invention provides a method for preventing a mammal in diseases associated with dendritic cell activity/maturation, by administering to the mammal a therapeutically effective amount VN of the present invention. Administration of the VN may occur prior to the manifestation of symptoms characteristic of the disease, such that the disease is prevented or, alternatively, delayed in its progression.

The present invention further relates to a pharmaceutical composition comprising the VN and a pharmaceutically acceptable carrier. The pharmaceutical composition may alternatively be administered subcutaneously, parenterally, intravenously, intradermally, intramuscularly, transdermally, intraperitoneally, or by inhalation or mist-spray delivery to lungs.

The carrier can be a solvent or dispersion medium containing, for example, water, ethanol, polyol (e.g., glycerol, propylene glycol, and liquid polyethylene glycol, and the like), or suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered, if necessary, and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The microparticles of the present invention may also be administered into the epidermis using the Powderject System (Chiron, Corp. Emeryville, Calif.). The Powderject's delivery technique works by the acceleration of fine particles to supersonic speed within a helium gas jet and delivers pharmaceutical agents and vaccines to skin and mucosal injection sites, without the pain or the use of needles.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

The phrase “pharmaceutically-acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

The term “therapeutically effective amount” as used herein, is that amount achieves, at least partially, a desired therapeutic or prophylactic effect in an organ or tissue. The amount of the VN necessary to bring about prevention and/or therapeutic treatment of the dendritic cell activation/maturation related diseases (such as infectious diseases, cancers and autoimmune diseases) or conditions is not fixed per se. An effective amount is necessarily dependent upon the identity and form of VN employed, the extent of the protection needed, or the severity of the diseases or conditions to be treated.

The treatment schedule and dosages may be varied on a subject by subject basis, taking into account, for example, factors such as the weight and age of the subject, the type of disease being treated, the severity of the disease condition, previous or concurrent therapeutic interventions, the manner of administration and the like, which can be readily determined by one of ordinary skill in the art.

For example, when used as a vaccine, the VN is administered in a manner compatible with the dosage formulation, and in such amount as will be therapeutically effective and immunogenic. The quantity to be administered depends on the subject to be treated, including, e.g., the capacity of the individual's immune system to synthesize antibodies, and the degree of protection desired. The dosage of the vaccine will depend on the route of administration and will vary according to the size of the host. Precise amounts of an active ingredient required to be administered depend on the judgment of the practitioner. In certain embodiments, pharmaceutical compositions may comprise, for example, at least about 0.1% of an active compound. In other embodiments, an active compound may comprise between about 2% to about 75% of the weight of the unit, or between about 25% to about 60%, for example, and any range derivable therein However, a suitable dosage range may be, for example, of the order of several hundred micrograms active ingredient per vaccination. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per vaccination, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above. A suitable regime for initial administration and booster administrations (e.g., inoculations) are also variable, but are typified by an initial administration followed by subsequent inoculation(s) or other administration(s).

In many instances, it will be desirable to have multiple administrations of the vaccine, usually not exceeding six vaccinations, more usually not exceeding four vaccinations and preferably one or more, usually at least about three vaccinations. The vaccinations will normally be at from two to twelve week intervals, more usually from three to five week intervals. Periodic boosters at intervals of 1-5 years, usually three years, will be desirable to maintain protective levels of the antibodies.

The course of the immunization may be followed by assays for antibodies for the supernatant antigens. The assays may be performed by labeling with conventional labels, such as radionuclides, enzymes, fluorescents, and the like. These techniques are well known and may be found in a wide variety of patents, such as U.S. Pat. Nos. 3,791,932; 4,174,384 and 3,949,064, as illustrative of these types of assays. Other immune assays can be performed and assays of protection from challenge with the immunostimulatory peptide can be performed, following immunization.

Currently, the most successful vaccines are typically live or attenuated pathogens-due in part to the cascade of events triggered at a site of pathogen invasion that lead to the activation of specific programs of dendritic cell function (for optimal T cell activation) and the efficient transport of antigen to host lymph nodes for B cell activation. The life cycle of dendritic cells after immunization with live or attenuated organisms (and during natural infections) proceeds by a four step process that leads to the generation of effector and memory lymphocytes (illustrated in FIG. 3A [Cyster et al., J Exp Med., 189:447-50 (1999); Kimber et al., Br J Dermatol., 142:401-12, (2000)]. 1) Dendritic cells and their precursors are recruited to the site of infection from the surrounding tissue and blood via chemokines released at the site [McWilliam et al., J Exp Med., 179:1331-6 (1994); Sallusto et al., Eur J Immunol., 29:1617-25 (1999)]. 2) Recruited cells take up antigen (in both MHC class I and MHC class II pathways) [Banchereau, et al., Nature, 392:245-52 (1998); Banchereau, et al., Annu Rev. Immunol., 18:767-811 (2000)]. 3) Antigen-loaded cells receive maturation signals, triggering upregulation of costimulatory molecules and altering expression of chemokine receptors [Kabashima et al., Nat Med., 9:744-9 (2003)]. 4) DCs emigrate to the lymph nodes to initiate T cell activation [Vermaelen et al., J Exp Med., 193:51-60 (2001)].

The present invention is further illustrated by the following examples which should not be construed as limiting. The contents of all references, patents and published patent applications cited throughout this application, as well as the Figures and Tables are incorporated herein by reference.

### EXAMPLE 1

### In Vitro and In Vivo Characterization of Chemokine (MIP-3α) Controlled Release Microspheres

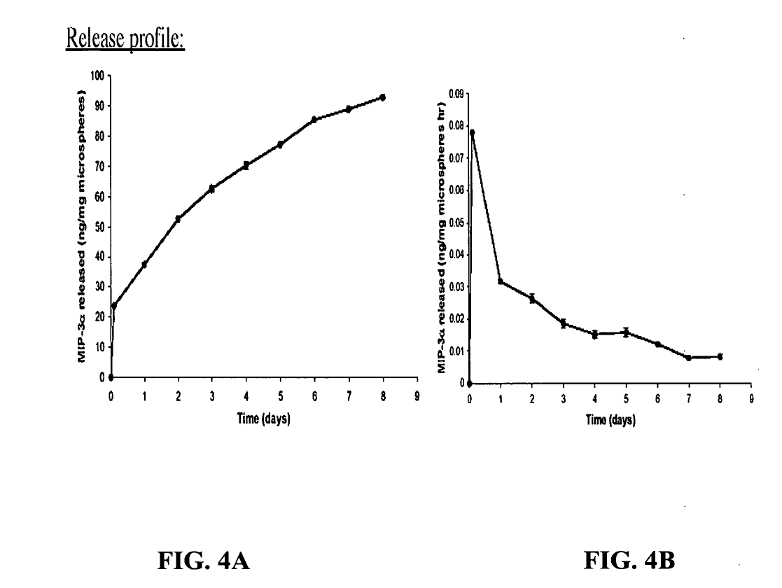

MIP-3α controlled release microspheres were synthesized to provide a steady gradient of this chemoattractant in vivo toward an immunization site. MIP-3α (R&D Systems) was encapsulated in poly(lactide-co-glycolide) microspheres by a double emulsion process as previously described [Lavelle et al., Nat Biotechnol., 20:64-9 (2002)]. To control release kinetics, microspheres were fabricated using PLGA having molecular weights 4.4 KDa or 75 KDa (Alkermes), which degrade at 37° C. in saline over a time course of 1-2 weeks and 3-4 weeks in vitro, respectively. (Release of encapsulated factors significantly precedes complete degradation of the polymer). Following prior reports [Kumamoto et al., Nat Biotechnol., 20:64-9 (2002); Kim et al., Biomaterials, 18:1175-84 (1997)], BSA was used as a carrier protein to protect the chemokines during encapsulation. Release profiles were measured in vitro by enzyme-linked immunosorbent assay (ELISA, R&D systems) on supernatant of microsphere samples incubated in PBS pH 7.4 at 37° C. to detect released chemokines, and showed release kinetics as shown in FIG. 4.

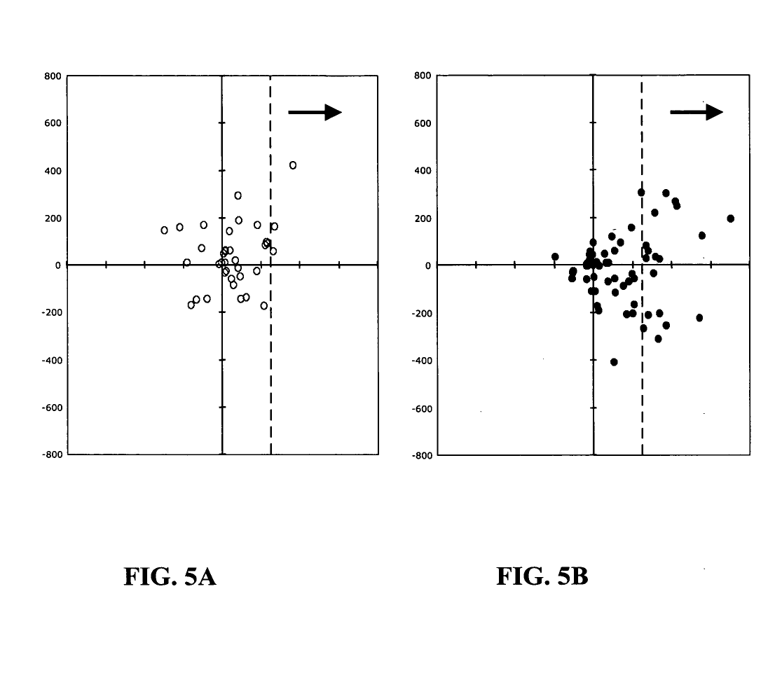

The controlled release microspheres were tested to determine whether they could attract immature bone marrow-derived dendritic cells in 3D collagen matrices, by performing videomicroscopy experiments monitoring the migration of DCs added to gels containing 0.1-1 mg of microspheres in a central well. Shown in FIG. 5 is an example of results from two different experiments, where the endpoints of individual cells are plotted, relative to their starting point at x=0, y=0. The direction toward the source microspheres is denoted by the arrow. Points in FIG. 5A indicate cells that started within 500 μm of the source microspheres, while the black points (FIG. 5B) indicate cells that were located approximately 500-1200 μm from the source. After 3 hours, significant migration of DCs toward the microspheres was observed.

Briefly, bone marrow-derived murine dendritic cells were suspended in collagen gels surrounding a well containing control or MIP-3α-releasing microspheres. Shown are 2D plots of path endpoints; each cell's starting point resides at origin, points indicate cell's location after 8 hour incubation. Arrows denote direction toward microsphere source. Shown at left is the response to control ‘empty’ microspheres, and at right, the response to MIP-3α-releasing microspheres.

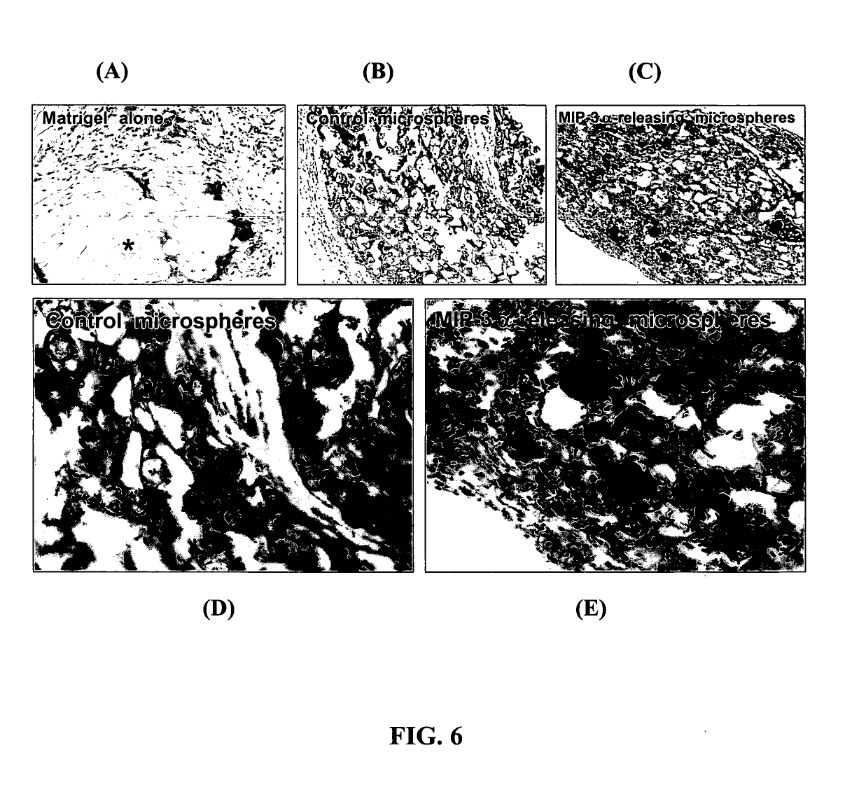

Next, the MIP-3α microspheres were tested for their chemoattractant properties in vivo (FIG. 6). Mice were implanted with matrigel alone, matrigel+control microspheres containing BSA, or microspheres containing MIP-3α. Implant sites were harvested at 24 hrs and stained with H&E. MIP-3α microspheres induced significant infiltration of the matrigel matrix and accumulation of cells around individual microspheres as shown in the FIG. 6.

More generally, these results show that chemokine-loaded microspheres can be used to attract cells to a particular site in vitro and/or in vivo. These microparticles act as a “hub” or town assembly hall inducing various cells to migrate to the site. Once attracted to a particular site, cells can then be programmed to perform a certain function as discussed in Example 2

### EXAMPLE 2

### Preparation of Antigen Delivery/DC Maturation Hydrogel Particles

Ovalbumin (60 mg) was dissolved in 100 ml 5M sodium chloride solution in water. Even though OVA was used as the model antigen for the proof-of-concept demonstrations provided herein, any antigen or peptide could be encapsulated in the nanogels. This protein solution was stirred at 600 rpm for 30 min to allow ovalbumin to salt out and form an emulsion at 37° C. The co-monomers—poly(ethylene glycol) methacrylate (526 Da, 2 ml), 2-aminomethacrylate (50 mg), poly(ethylene glycol) dimethacrylate (875 Da, 200 μl), and 100 mg of PEG-peptide-PEG were slowly added to the protein solution and allowed to also salt out into the protein-rich phase. Initiators ammonium persulfate and sodium metabisulfite (200 μl of 10% w/vol APS and 10% w/vol SMS) were added to the same aqueous medium followed by reaction at 40° C. for 5-30 min. The particles were separated by centrifuging the suspension at 10,000 rpm for 15 min and washing with water twice. Finally, the gel particles thus obtained were suspended in PBS and lyophilized. The particles were stored at 4° C. until use.

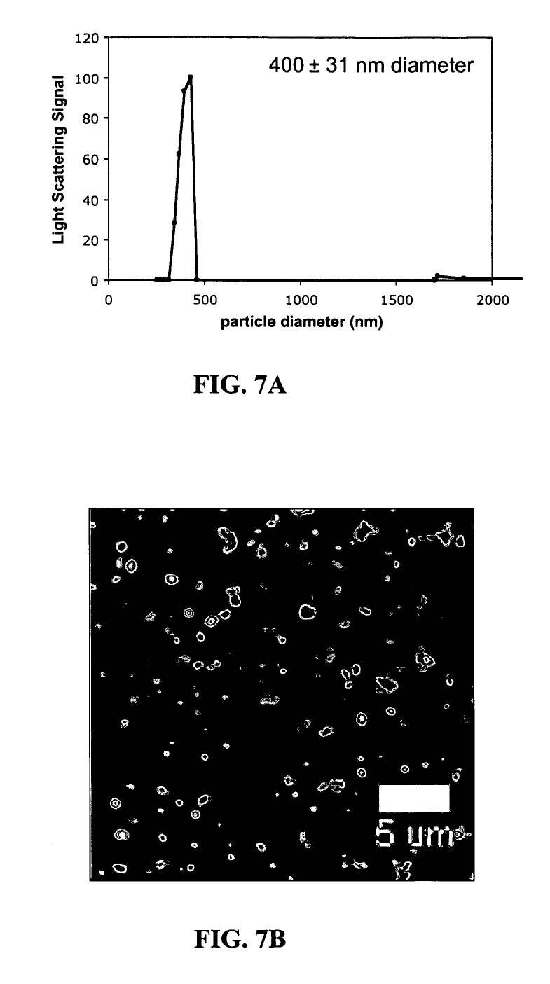

The size and size distribution of the synthesized hydrogel particles will be determined by photon correlation spectroscopy (Brookhavens 90Plus). Protein loading in microgels was estimated by the BCA colorimetric assay (Pierce Chemical Co.). Sizing and loading data are shown in FIG. 7 and Table 1

TABLE 1
Particle Characteristics

Particle Composition Data

| Total protein loading | 738.26 μg ova per mg particles |
|---|---|
| Encapsulation efficiency | 49.22% |
| Protein released from particles at 4 days | 8.80% |

### EXAMPLE 3

### In Vitro Antigen Delivery to Dendritic Cells Using Hydrogel Particles

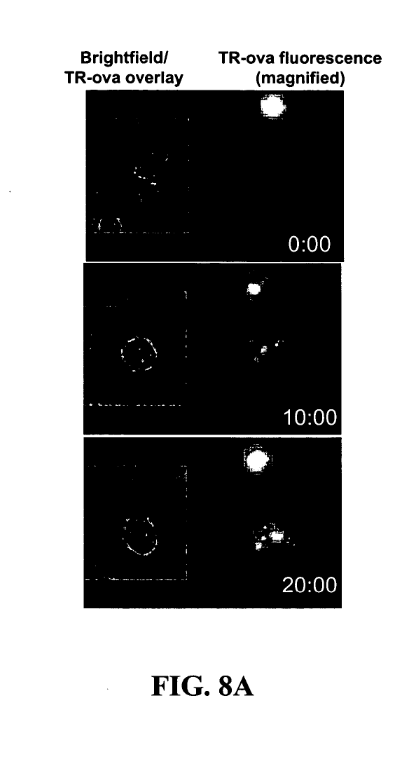

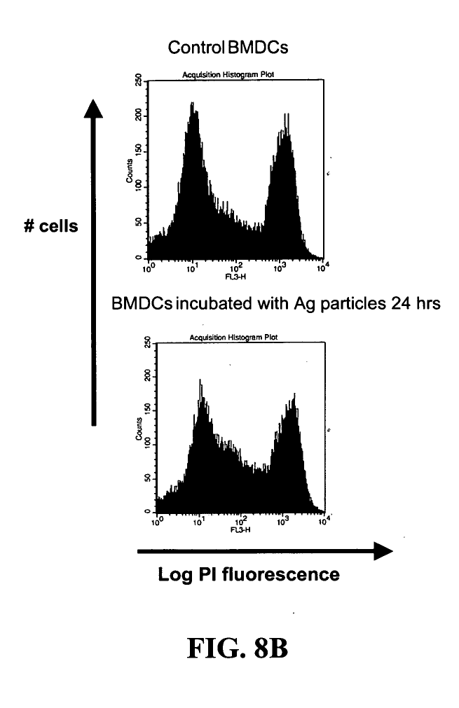

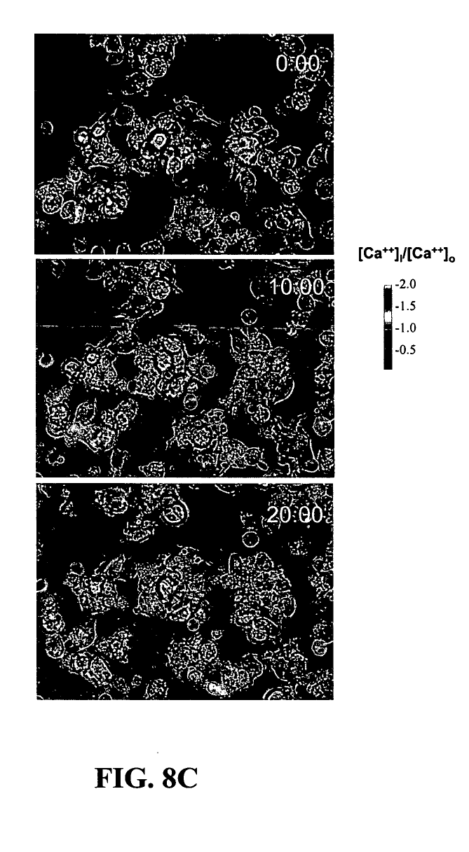

Bone marrow-derived dendritic cells were generated in the presence of GM-CSF and IL-4 as previously described [Inaba et al., J Exp Med., 176:1693-702 (1992)]. To assess antigen-loaded gel particle uptake, time-lapse 3D fluorescence microscopy was performed on live DC cultures to which 5 μg/ml Texas red-ovalbumin-loaded particles had been added. FIG. 8A shows three frames of a representative DC showing the internalization of particles over the first 20 min of culture. DCs efficiently phagocytosed the antigen-loaded gel particles from the surrounding solution. To assay for cytotoxicity of internalized gel particles, a known amount of gels (1 μg particles) were incubated with 2×10⁵ DCs in 250 μl medium for 20 hrs followed by a change of media. As shown in FIG. 8B, particle-treated DCs and controls that were subsequently stained with propidium iodide and analyzed by flow cytometry to detect changes in the relative proportions of live cells in the cultures detected no significant effect of the gel particle uptake on DC viability.

MHC class I presentation that is critical for cell-mediated immunity was detected by activation of a CD8⁺ cytotoxic T cell (CTL) clone in vitro. Day 7 bone marrow-derived DCs (2×10⁵ in 250 μL medium) were incubated with 5 μg/ml ovalbumin-loaded particles 20 hrs, washed to remove non-internalized particles, then 5×10⁴ 4G3 CD8⁺ T cells loaded with a calcium indicator fluorescent dye (fura-2AM) were added to particle-treated DCs or untreated controls. The 4G3 T cell clone recognizes a peptide fragment from ovalbumin on class I MHC. Time-lapse fluorescence microscopy was performed to track interactions of the T cells with DCs over 4 hrs. T cells on controls migrated over DCs but did not form lasting contacts with single cells and did not elevate their intracellular calcium levels. In contrast, in the example of time-lapse frames as shown in FIG. 8C, T cells interacting with particle-treated DCs were induced to stop migration and form long-lived contacts with DCs, and fluxed calcium, as indicated by the change of the fura indicator false-color fluorescence from purple background levels. By 8 hrs of culture, the majority of the DCs in the culture had been killed by the activated cytotoxic T cells, while DCs in the control culture remained healthy. This data indicates that the antigen delivery gel particles were able to successfully deliver exogenous ovalbumin antigen to the MHC class I antigen presentation pathway, a key requirement for successful cancer or intracellular pathogen vaccines.

### EXAMPLE 4

### Processing and Release of Antigen from Antigen Delivery Particles

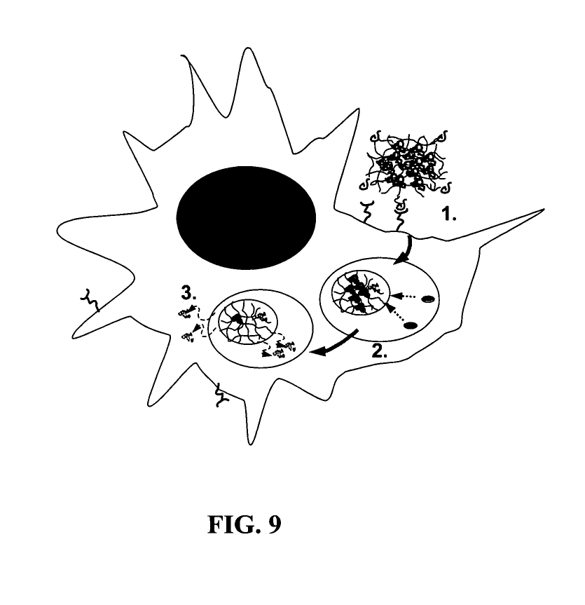

FIG. 9 shows one possible mechanism for antigen processing and presentation to both MHC I and MHC II molecules from hydrogel particles, which comprise a polymer mesh surrounding the protein antigen. Briefly, proteases small enough to diffuse through the gel particle mesh may enter the gel particles, proteolyze the entrapped protein antigen, and the resulting protein fragments may subsequently diffuse out of the particles to be processed by the normal intracellular antigen processing pathways. Alternatively, protein cross-linked to the polymer near the surface of the particles may be accessed by proteases at the surface, subsequently creating space for entry of proteases into the particles. Evidence for these mechanisms was obtained by in vitro studies where we incubated the ova-containing gel particles with purified cathepsin D, a protease present in the endosomes of dendritic cells and which is known to proteolyze ovalbumin.

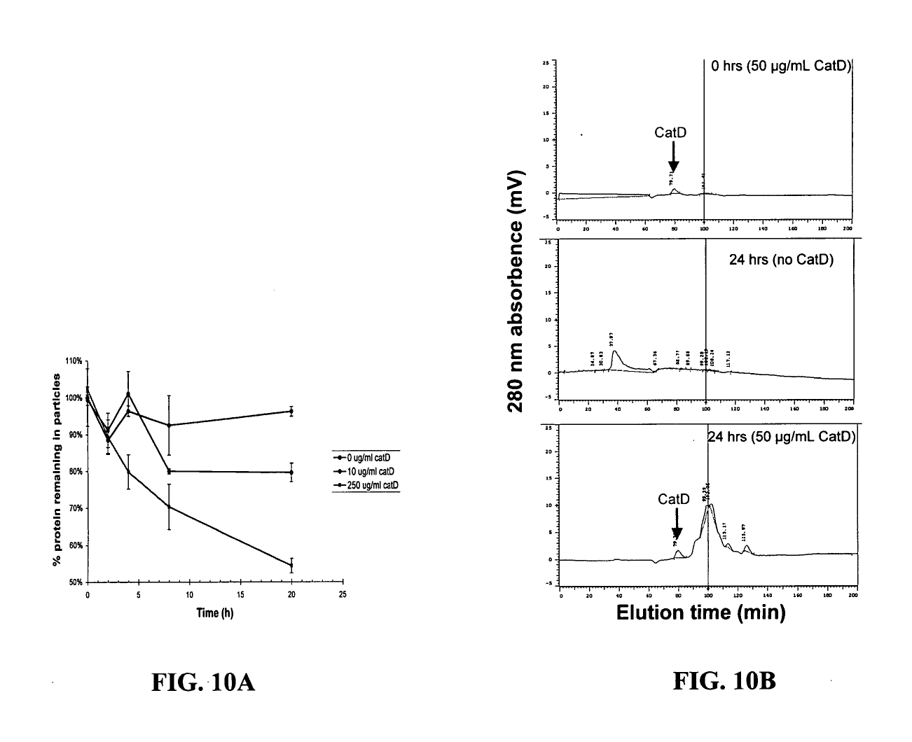

As shown in FIG. 10, cathepsin D caused the loss of protein from ova particles in a dose dependent manner over time. Ova-loaded antigen delivery gel particles were incubated with varying doses of cathepsin D in pH 5.5 buffer mimicking conditions within endosomes. After the denoted times, particles were recovered by centrifugation and assayed for the content of protein remaining in the particles (FIG. 10A). Further, analysis of the supernatant of cathepsin D-treated particles by gel permeation chromatography showed that the protein released from particles was, in fact, proteolyzed to low molar mass fragments. As shown in FIG. 10B, at time 0 in the presence of cathepsin, only cathepsin is observed in the FPLC trace, as the particles are too large to pass the FPLC column pre-filter. After 24 hrs, prominent low molecular weight fragments appear in the chromatogram in the presence but not the absence of cathepsin D, indicating that cathepsin is degrading the ova entrapped in gel particles.

### EXAMPLE 5

### Maturation Signal Presentation: Plasticity of Dendritic Cells

DCs are capable of evolving from immature, antigen-capturing cells to mature, antigen-presenting, T cell-priming cells; converting antigens into immunogens and expressing molecules such as cytokines, chemokines, and costimulatory molecules to initiate an immune response. The types of T cell-mediated immune responses (tolerance vs. immunity, Th₁ vs. Th₂) induced can vary, however, depending on the specific DC lineage (myeloid DC1s or lymphoid DC2s) and maturation stage in addition to the activation signals received from the surrounding microenvironment [McColl et al., Immunol Cell Biol., 80:489-96 (2002); Sozzani et al., J Clin Immunol 20:151-60 (2000); Vermaelen et al., J Exp Med., 193:51-60 (2001)]. This ability of DCs to regulate immunity is dependent on DC maturation. A variety of factors can induce maturation following antigen uptake and processing within DCs, including: whole bacteria or bacterial-derived antigens (e.g. lipopolysaccharide, LPS), inflammatory cytokines, various small molecules, ligation of select cell surface receptors (e.g. CD40) and viral products (e.g. double-stranded RNA). The process of DC maturation, in general, involves a redistribution of major histocompatibility complex (MHC) molecules from intracellular endocytic compartments to the DC surface, down-regulation of antigen internalization, an increase in the surface expression of costimulatory molecules, morphological changes (e.g. formation of dendrites), cytoskeleton re-organization, secretion of chemokines, cytokines and proteases, and surface expression of adhesion molecules and chemokine receptors.

DCs are exquisitely sensitive to the stimulus that they encounter. By measuring the gene expression profiles of dendritic cells for ˜30,000 genes after encounter with influenza virus, E. coli, S. aureus, C. albicans and other pathogens [Huang et al., Science, 294:870-5 (2001)]. It was demonstrated that there is a shared response of dendritic cells to all pathogens and pathogen components, and there is also a highly specialized transcriptional response which is pathogen-specific. This specialized response at the transcriptional level leads to precise functional consequences in the type of immune response induced in vitro and in vivo. In parallel, apoptotic cells block the activation of T cells by dendritic cells and represent a form of ‘self’ that acts as an endogenous block of immunity.

To demonstrate the coupling of a model protein ligand to the surface of gel particles prepared by the procedure of present invention, fluorochrome-labeled ovalbumin was linked to the surface of ovalbumin-loaded gel particles. Ovalbumin-loaded particles were prepared as above, but incorporating, additionally, 100 mg 2-aminoethymethacrylate in the monomers. The particles were purified as before, then 250 μg Texas red-labeled ovalbumin was added to the particles and the suspension was shaken at 20° C. for 2 hrs. To covalently couple the adsorbed protein to the particle surfaces, 100 mg EDC carbodiimide was added to the suspension and the particles were shaken 10 hrs at 37° C. The gels were then pelleted by centrifugation and washed 3× with phosphate buffered saline. Measurement of the protein remaining in the supernatant from the washes with the BCA protein assay indicated 39% efficiency in the coupling of TR-ova to the surface of the particles. Protein coupling was confirmed by observation of the particles by fluorescence microscopy (data not shown).

Next, CpG DNA oligonucleotides were immobilized on the surface of the particles, as illustrated in FIG. 1. These surface-bound ligands have two functions: (1) they provide targeting of the particles to DCs, which specifically express receptors for CpG (and if desired, other activation factors), and (2) they trigger maturation of DCs once internalized in phagosomes, where they bind to TLR-9 receptors. Maturation of DCs induces the transport of internalized antigen to the surface in MHC molecules and causes the upregulation of cytokines and costimulatory receptors that drive T cell activation. Finally, maturation also induces expression of chemokine receptors that guide DCs to the host lymph nodes.

Even though CpG oligos were used as the maturation ligand, other maturation signal proteins discussed earlier can also be tethered to the hydrogel particles to program the DCs to elicit the desired immune response.

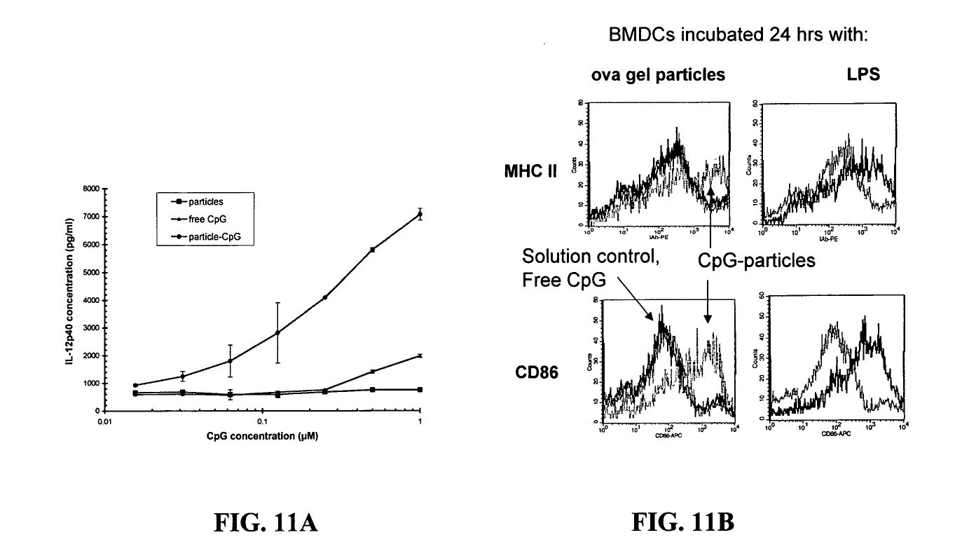

The maturation and activation of dendritic cells in response to antigen delivery particles with or without immobilized CpG were measured to determine the effect of this ligand on dendritic cell function. The production of the Th1 cytokine interleukin-12 by dendritic cells incubated with particles was used as an indicator of DC activation. As shown in FIG. 11A, bone marrow derived from immature dendritic cells (BMDCs) were not triggered to produce IL-12 by unmodified gel particles, consistent with their synthetic structure. However, soluble CpG triggers IL-12 production, particularly once the solution concentration approaches 1 mM. In contrast, CpG oligonucleotides immobilized to the surface of antigen delivery particles were ˜10-fold more potent than the same amount of soluble CpG in triggering DC activation. CpG is also known to trigger upregulation of MHC molecules and costimulatory molecules such as CD86 as immature DCs are triggered to mature. Flow cytometry analysis of cell surface levels of class II MHC and CD86 are shown in FIG. 11B for immature BMDCs exposed to free CpG or equivalent concentrations of CpG bound to antigen delivery particles for 24 hrs. Soluble CpG showed little or no effect on BMDC maturation under these conditions, while CpG-particles triggered robust upregulation of both cell surface molecules, comparable to the strong stimulatory control (BMDCs incubated with lipopolysaccharide (LPS)). Thus, the antigen delivery particles do not activate DCs intrinsically, but when modified with a selected DC-modulatory ligand, they can initiate DC activation and maturation much more potently than simple application of soluble ligands.

### EXAMPLE 6

### Comparison of Particle Delivered Antigen Vs. Soluble Antigen for T Cell Activation

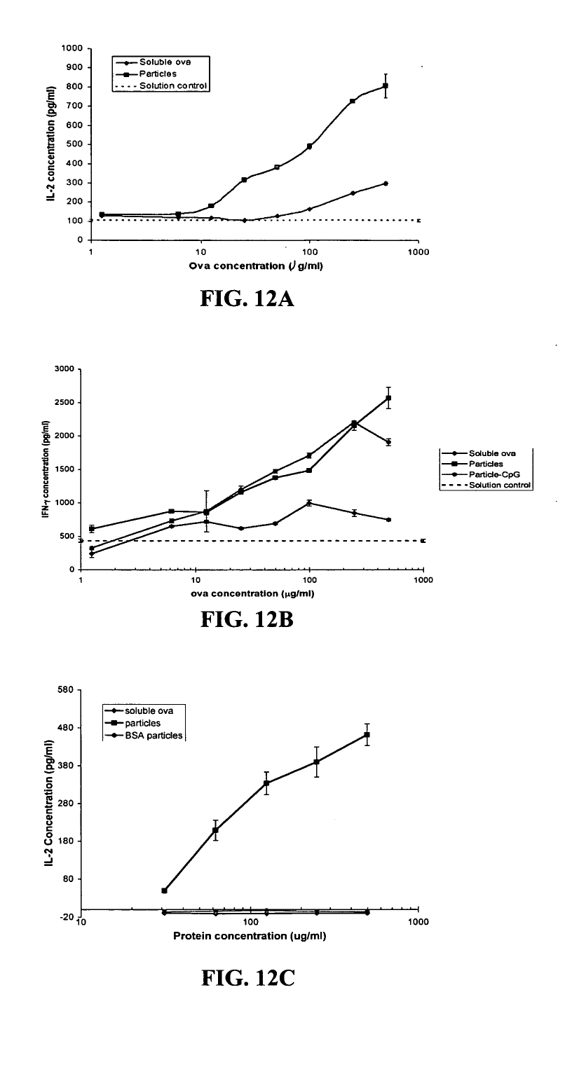

As described earlier, dendritic cells pulsed with antigen delivery particles effectively processed and presented antigen, as assessed by the activation of CD4⁺ T cell blasts and CD8⁺ T cell clones. As shown in FIG. 12A, BMDCs pulsed with ova particles activated CD4⁺ ova-specific T cells, and particles were ˜10 fold more potent than soluble ovalbumin at activating CD4 cells. Particle-pulsed DCs triggered CD4 cells to produce significant levels of the Th1 effector cytokine interferon-γ (IFN-γ), with or without CpG bound to the particles (FIG. 12B). More dramatic is the impact of particle-based delivery of antigen for activation of CD8 T cells (FIG. 12C). As demonstrated in numerous previous studies, soluble ovalbumin is not presented on class I MHC, and thus BMDCs pulsed with soluble ova fail to trigger CD8 T cell activation. In contrast, ova delivered in gel particles primed strong CD8 T cell activation. This activation was specific to the antigen, as particles encapsulating an irrelevant antigen (bovine serum albumin) failed to trigger CD8 responses.

In summary, ova particles are highly efficient at delivering antigen to both class I and class II MHC pathways, and potently activate primary T cells (both CD4 and CD8 T cells). T cells are activated to a Th1-like response and produce effector cytokines.

### EXAMPLE 7

### Activation of Naive CD4⁺ and CD8⁺ T Cells In Vitro and In Vivo Using Hydrogel Antigen Delivery/DC Activation Particles

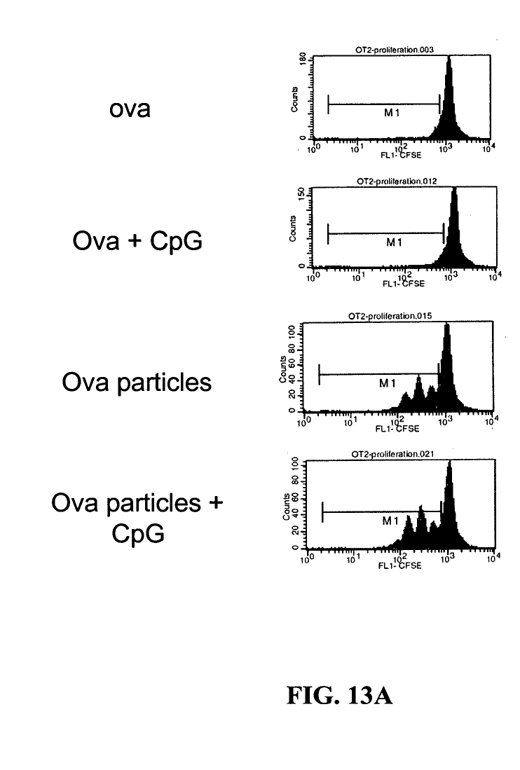

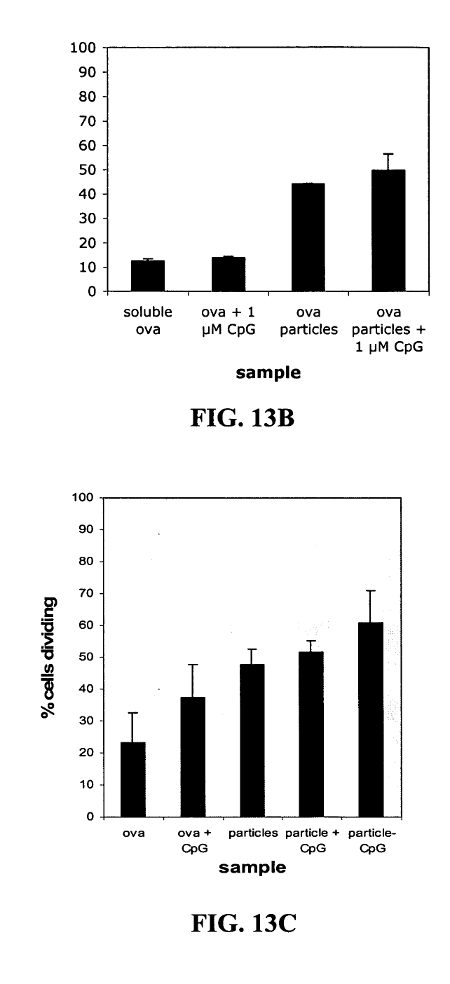

Murine bone marrow-derived dendritic cells were loaded with antigen by incubation with 50 μg ovalbumin either in soluble form or encapsulated in hydrogel delivery particles, in the presence or absence of 1 μM CpG for 4 hours. DCs were then cultured with carboxyfluorescien succinimidyl ester (CFSE)-loaded CD4⁺ OT-II or CD8⁺ OT-1 T cells for 60 hours. CFSE is a fluorescent dye that labels the cytoplasm of the cells; when the cells divide, the dye is divided approximately equally between the two daughter cells, having the total fluorescence in the daughter cells. Using this labeling technique, cell division is readily quantified by flow cytometric analysis of the T cells. FIG. 13 shows such an analysis for OT-1 and OT-II T cells responding to DCs pulsed with soluble ova or ova encapsulated in gel particles. FIG. 13A shows the flow cytometry histograms plotting the percentage of OT-II T cells detected using CFSE fluorescence. Dendritic cells that were pulsed with soluble antigen or soluble antigen plus CpG, almost no cell division occurred by 60 hours, and no cells divided more than one time. In contrast, DCs pulsed with ova particles triggered significant cell division and many cells had already divided 3-4 times by 60 hours. As shown in FIG. 13B, the percentages of cells that had divided under each condition is quantified, and the significant increase in T cell responses elicited with the antigen delivery particles. Similar experiments carried out with OT-1 CD8⁺ T cells showed that antigen delivery particles also promoted cross-presentation and activation of naive CD8⁺ cells, as shown in FIG. 13C. Roughly twice as many CD8⁺ T cells had divided by 60 hours when ova-CpG-particle-pulsed DCs were used to present antigen, in comparison to DCs pulsed with soluble ova and CpG. Thus, consistent with our earlier experiments on T cell blasts, antigen delivery particles drive significantly more potent activation of naive T cells (both CD4⁺ and CD8⁺) when compared to soluble antigen, even in the presence of soluble CpG as an adjuvant.

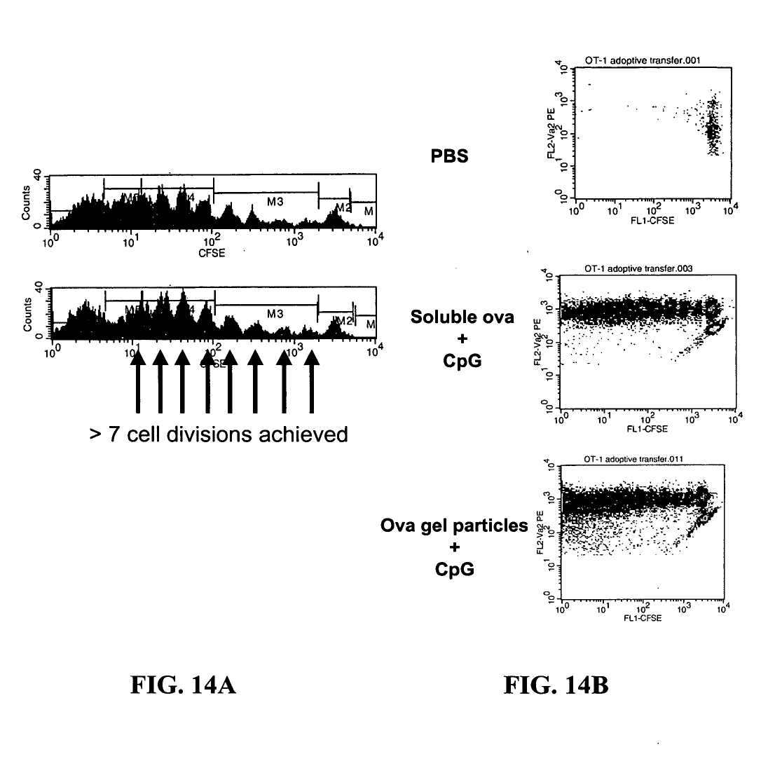

CFSE-labeled T cells (OT-I or OT-II, in separate experiments) were then adoptively transferred into wild type B6 mice and allowed to home to secondary lymphoid organs for 24 hours. Mice were then immunized with either soluble ova or ova gel particles in the presence or absence or CpG. Five days later, T cell responses to the immunization were assayed by analyzing T cells present in the draining lymph nodes by flow cytometry. As shown in FIG. 14, both CD4⁺ and CD8⁺ naive T cells were activated and showed extensive proliferation in vivo in response to the gel particle immunization. Shown at left are CFSE fluorescence histograms of OT-II T cells recovered from 2 different mice immunized with ova gel particles, showing up to 7 or more divisions by some cells, and significant expansion of the total population. At right are scatter plots showing responses of OT-I T cells: TCR expression levels on the vertical axis and CFSE fluorescence on the horizontal axis, for a control (PBS injection), soluble ova plus CpG, and ova particles plus CpG. As expected, no cell division is seen for the control injection. Soluble ova mixed with soluble CpG at the given (high) antigen dose triggered significant OT-I T cell proliferation (in agreement with other published data on OT-I T cells). However, immunization with gel particles triggered an even greater T cell response, as evidenced by the further shifting of T cell CFSE fluorescence toward the origin-an indication of maximal cell division occurring.

In summary, OVA encapsulated particles are highly efficient at delivering antigen to both class I and class II MHC pathways, and potently activate primary T cells (both CD4⁺ and CD8⁺ T cells). T cells are activated to a Th1-like response and produce effector cytokines. Furthermore, this data indicates that the antigen delivery/DC activation system leads to potent naive T cell activation in vitro, and also functions to prime T cells in vivo.

### EXAMPLE 8

### Fabrication and Characterization of Colloidal Micelles

To integrate the chemoattraction microspheres and antigen delivery/DC activation particles, conjugated ‘colloidal micelles’ of these two components (as illustrated in FIG. 3B) can be synthesized by forming temporary covalent linkages of the particle gels to the surface of microspheres, the PLGA spheres are treated with 1M NaOH for 15 min to induce surface layer hydrolysis and the introduction of carboxylate groups. The microspheres are then washed 3× to remove the base. If base treatment is found to significantly alter microsphere release kinetics or other physical properties, carboxylate end-capped PLGA (Boeringer Ingleheim) [Faraasen et al., Pharm Res., 20:237-46 (2001)] can be used as an alternative.

Briefly, loaded antigen delivery/DC activation particles are coupled to the carboxy-modified microspheres via carbodiimide coupling, utilizing free amines remaining on the surface of the particles. Microspheres at a concentration of 5×10⁶ particles/ml are mixed with gel particles at a 1:200 microsphere:particle ratio and allowed to equilibrate for 15 minutes with agitation. Subsequently, the water-soluble carbodiimide EDC is added (5 mM) and the spheres are permitted to react for 2 hrs at room temperature with agitation. At the end of the incubation period, the microspheres with bound particles are separated from unbound nanospheres by brief centrifugation at 1000×g for 5 min. Similar particle concentrations have been previously reported to provide a high yield of colloidal micelles [Huang et al., Science, 294:870-5 (2001)]. Colloidal micelles are finally washed several times to remove residual carbodiimide and urea byproducts, then lyophilized and stored at 4° C. until used.

The preferred embodiments of the compounds and methods of the present invention are intended to be illustrative and not limiting. Modifications and variations can be made by persons skilled in the art in light of the above teachings. It is also conceivable to one skilled in the art that the present invention can be used for other purposes of measuring the acetone level in a gas sample, e.g. for monitoring air quality. Therefore, it should be understood that changes may be made in the particular embodiments disclosed which are within the scope of what is described as defined by the appended claims.

1. A composition for modulating an immune response against an antigen in a mammal, said composition comprising a hydrogel particle, wherein said hydrogel particle comprises:
a hydrogel polymer;
an immunogen encapsulated in said hydrogel particle; and
a ligand on a surface of said hydrogel particle, said ligand interacts with an antigen presenting cell and providing an activation signal to said antigen presenting cell.

2. The composition of claim 1, wherein said immunogen is selected from the group consisting of a biopolymer, a cell lysate, and a synthetic antigen.

3. The composition of claim 2, wherein said biopolymer is selected from the group consisting of polypeptide, polynucleotide, polysaccharide, lipid, and a mixture thereof.

4. The composition of claim 1, wherein said immunogen comprises at least one of a bacterial antigen, a viral antigen, a parasitic antigen, a tumor-specific antigen, a tissue graft antigen, a self-antigen, a synthetic antigen, and an allergen.

5. The composition of claim 1, wherein said hydrogel polymer comprises polyethylene glycol [PEG] methacrylate and acrylates, poly(acrylic acid), poly(methacrylic acid), 2-diethylaminoethylmethacrylate, 2-aminoethyl methacrylate, poly(ethylene glycol) dimethacrylates and acrylates, acrylamide/bisacrylamide, poly(2-hydroxyethyl methacrylate), methacrylated dextrans, acrylated dextrans, or poly(ethylene glycol)-polyester acrylated/methacrylated block copolymer.

6. The composition of claim 1, wherein said ligand is covalently attached to the surface of said hydrogel particle.

7. The composition of claim 1, wherein said ligand is non-covalently attached to the surface of said hydrogel particle.

8. The composition of claim 1, wherein said ligand is selected from the group consisting of CpG, CD40 ligand, vitamin D, dsRNA, poly(I:C), IL-2, IL-4, IL-7, IL-13, IL-15, LPS, bacterial lipoproteins, lipid A, TGF-β, TLR7 ligands (imidazoquinolines), antibodies against TLR receptors, and antibodies against DEC-205.

9. The composition of claim 1, wherein said hydrogel particle further comprises enzyme-sensitive or environment-sensitive polymer sequences that permit the selective release of said encapsulated biopolymer upon delivery of said hydrogel particle to an intracellular compartment or extracellular matrix.

10. The composition of claim 1, wherein said hydrogel particle has an average diameter of 10 nm-50 μm.

11. The composition of claim 1, further comprising a microsphere, wherein said microsphere comprises a chemoattractant.

12. The composition of claim 11, wherein said chemoattractant is a cytokine.

13. The composition of claim 12, wherein said cytokine is selected from the group consisting of IL-12, IL-1α, IL-1β, IL-15, IL-18, IFNα, IFNβ, IFNγ, IL-4, IL-10, IL-6, IL-17, IL-16, TNFα, and MIF.

14. The composition of claim 11, wherein said chemoattractant is a chemokine.

15. The composition of claim 14, wherein said chemokine is selected from the group consisting of MIP-3a, MIP-1a, MIP-1b, RANTES, MIP-3b, SLC, fMLP, IL-8, SDF-1α, and BLC.

16. The composition of claim 1, wherein said antigen presenting cell is a dendritic cell.

17. A pharmaceutical composition comprising:
the composition of claim 1; and
a pharmaceutically acceptable carrier.

18. The pharmaceutical composition of claim 17, wherein said composition further comprises a microsphere contains a chemoattractant.

19. The pharmaceutical composition of claim 18, wherein said hydrogel particle and said microsphere are conjugated to form a colloidal micelle.

20. The pharmaceutical composition of claim 19, wherein said hydrogel particle is conjugated to said microsphere via carbodiimide coupling.

21. An antigen delivery system for both antigen presentation and dendritic cell activation, comprising:
a hydrogel particle, and
a microsphere,
wherein said hydrogel particle comprises:
a hydrogel polymer;
an immunogen encapsulated in said hydrogel particle; and
a ligand on a surface of said hydrogel particle, said ligand interacts with a dendritic cell and providing an activation signal to said dendritic cell.

22. The antigen delivery system of claim 21, wherein said immunogen is selected from the group consisting of a biopolymer, a cell lysate, and a synthetic antigen.

23. The antigen delivery system of claim 22, wherein said biopolymer is selected from the group consisting of polypeptide, polynucleotide, polysaccharide, lipid, and a mixture thereof.

24. The antigen delivery system of claim 21, wherein said immunogen comprises at least one of a bacterial antigen, a viral antigen, a parasitic antigen, a tumor-specific antigen, a tissue graft antigen, a self-antigen, a synthetic antigen, and an allergen.

25. The antigen delivery system of claim 21, wherein said hydrogel polymer comprises polyethylene glycol [PEG] methacrylate and acrylates, poly(acrylic acid), poly(methacrylic acid), 2-diethylaminoethylmethacrylate, 2-aminoethyl methacrylate, poly(ethylene glycol) dimethacrylates and acrylates, acrylamide/bisacrylamide, poly(2-hydroxyethyl methacrylate), methacrylated dextrans, acrylated dextrans, or poly(ethylene glycol)-polyester acrylated/methacrylated block copolymer.

26. The antigen delivery system of claim 21, wherein said ligand is covalently attached to the surface of said hydrogel particle.

27. The antigen delivery system of claim 21, wherein said ligand is non-covalently attached to the surface of said hydrogel particle.

28. The antigen delivery system of claim 21, wherein said ligand is selected from the group consisting of CpG, CD40 ligand, vitamin D, dsRNA, poly(I:C), IL-2, IL-4, IL-7, IL-13, IL-15, LPS, bacterial lipoproteins, lipid A, TGF-β, TLR7 ligands (imidazoquinolines), antibodies against TLR receptors, and antibodies against DEC-205.

29. The antigen delivery system of claim 21, wherein said hydrogel particle further comprises enzyme-sensitive or environment-sensitive polymer sequences that permit the selective release of said encapsulated biopolymer upon delivery of said hydrogel particle to an intracellular compartment or extracellular matrix.

30. The antigen delivery system of claim 21 wherein said hydrogel particle has an average diameter of 10 nm-50 μm.

31. The antigen delivery system of claim 21, wherein said microsphere comprises a chemoattractant.

32. The antigen delivery system of claim 31, wherein said chemoattractant is a cytokine.

33. The antigen delivery system of claim 32, wherein said cytokine is selected from the group consisting of IL-12, IL-1α, IL-1β, IL-15, IL-18, IFNα, IFNβ, IFNγ, IL-4, IL-10, IL-6, IL-17, IL-16, TNFα, and MIF.

34. The antigen delivery system of claim 31, wherein said chemoattractant is a chemokine.

35. The antigen delivery system of claim 34, wherein said chemokine is selected from the group consisting of MIP-3a, MIP-1a, MIP-1b, RANTES, MIP-3b, SLC, fMLP, IL-8, SDF-1α, and BLC.

36. A pharmaceutical composition comprising:
said antigen delivery system of claim 21; and
a pharmaceutically acceptable carrier.

37. The pharmaceutical composition of claim 36, wherein said hydrogel particle and said microsphere are conjugated to form a colloidal micelle.

38. The pharmaceutical composition of claim 37, wherein said hydrogel particle is conjugated to said microsphere via carbodiimide coupling.

39. A method for enhancing an immune response to an antigen in a mammal, said method comprising:
administering to said mammal a therapeutically effective amount of a composition comprising a hydrogel particle which comprises:
a hydrogel polymer;
said antigen, or a polynucleotide encoding said antigen, encapsulated in said hydrogel particle; and
a ligand on a surface of said hydrogel particle, said ligand interacts with an antigen presenting cell; and
a pharmaceutically acceptable carrier.

40. The method of claim 39, wherein said antigen presenting cell is a dendritic cell.

41. The method of claim 39, wherein said composition further comprises a microsphere which comprises a chemoattractant.

42. The method of claim 41, wherein said chemoattractant comprises a cytokine or a chemokine.

43. The method of claim 39, wherein said antigen comprises at least one of a bacterial antigen, a viral antigen, a parasitic antigen, a tumor-specific antigen, and a synthetic antigen.

44. A method of suppressing immune response to an antigen in a mammal, said method comprising:
administering to said mammal a therapeutically effective amount of a composition comprising a hydrogel particle which comprises:
a hydrogel polymer;
said antigen, or a polynucleotide encoding said antigen, encapsulated in said hydrogel particle; and
a ligand on a surface of said hydrogel particle, said ligand interacts with an antigen presenting cell; and
a pharmaceutically acceptable carrier.

45. The method of claim 44, wherein said antigen presenting cell is a dendritic cell.

46. The method of claim 44, wherein said composition further comprises a microsphere which comprises a chemoattractant.

47. The method of claim 46, wherein the chemoattractant is a cytokine or chemokine.

48. The method of claim 44, wherein said antigen comprises at least one of a tissue graft antigen, a self-antigen, a synthetic antigen, and an allergen.

49. A method for treating an infectious disease, cancer or an autoimmune disease in a mammal, said method comprising:
administering to said mammal a therapeutically effective amount of the pharmaceutical composition of claim 17.

50. The method of claim 49, wherein said pharmaceutical composition is administered intramuscularly.

51. The method of claim 49, wherein said pharmaceutical composition is administered subcutaneously.

52. The method of claim 49, wherein said pharmaceutical composition is administered intradermally.

53. The method of claim 49, wherein said pharmaceutical composition is administered by a powderject system.

54. The method of claim 49, wherein said pharmaceutical composition is administered by inhalation or mist-spray delivery to lungs.

55. The method of claim 49, wherein said infectious disease is caused by at least one of microbe selected from the group consisting of Actinobacillus actinomycetemcomitans; Bacille Calmette-Gurin; Blastomyces dermatitidis; Bordetella pertussis; Campylobacter consisus; Campylobacter recta; Candida albicans; Capnocytophaga sp.; Chlamydia trachomatis; Eikenella corrodens; Entamoeba histolitica; Enterococcus sp.; Escherichia coli; Eubacterium sp.; Haemophilus influenzae; Lactobacillus acidophilus; Leishmania sp.; Listeria monocytogenes; Mycobacterium vaccae; Neisseria gonorrhoeae; Neisseria meningitidis; Nocardia sp.; Pasteurella multocida; Plasmodium falciparum; Porphyromonas gingivalis; Prevotella intermedia; Pseudomonas aeruginosa; Rothia dentocarius; Salmonella typhi; Salmonella typhimurium; Serratia marcescens; Shigella dysenteriae; Streptococcus mutants; Streptococcus pneumoniae; Streptococcus pyogenes; Treponema denticola; Trypanosoma cruzi; Vibrio cholera; and Yersinia enterocolitica.

56. The method of claim 49, wherein said infectious disease is caused by at least one of virus selected from the group consisting of influenza virus; parainfluenza virus; rhinovirus; hepatitis A virus; hepatitis B virus; hepatitis C virus; apthovirus; coxsackievirus; Rubella virus; rotavirus; Denque virus; yellow fever virus; Japanese encephalitis virus; infectious bronchitis virus; Porcine transmissible gastroenteric virus; respiratory syncytial virus; Human immunodeficiency virus (HIV); papillomavirus; Herpes simplex virus; varicellovirus; Cytomegalovirus; variolavirus; Vacciniavirus; suipoxvirus; and coronavirus.

57. The method of claim 56, wherein said infectious disease is caused by HIV.

58. The method of claim 49, wherein said cancer is breast cancer, colon-rectal cancer, lung cancer, prostate cancer, skin cancer, osteocarcinoma, or liver cancer.

59. The method of claim 49, wherein said autoimmune disease is asthma, systemic lupus erythematosus (SLE), rheumatoid arthritis, multiple sclerosis, juvenile-onset diabetes, autoimmune uveoretinitis, autoimmune vasculitis, bullous pemphigus, myasthenia gravis, autoimmune thyroiditis or Hashimoto's disease, Sjogren's syndrome, granulomatous orchitis, autoimmune oophoritis, Crohn's disease, sarcoidosis, rheumatic carditis, ankylosing spondylitis, Grave's disease, or autoimmune thrombocytopenic purpura.

60. The method of claim 59, wherein said autoimmune disease is asthma or SLE.

61. A method for producing the composition set forth in claim 1, comprising the steps of:
(a) preparing a solution containing an immunogen;
(b) adding a salt to said immunogen solution to salt out and form an emulsion;
(c) adding a hydrogel monomer of said hydrogel to said emulsion to form an aqueous medium; and
(d) adding initiators to said aqueous medium to form a hydrogel particle.

62. The method of claim 61, wherein said immunogen is a biopolymer or a cell lysate.

63. The method of claim 61, wherein in step (d), said initiators is added to said aqueous medium under stirring to form said hydrogel particle.

64. The method of claim 62, wherein in step (b), said adding salt to said biopolymer solution to salt out and form said emulsion at 37° C.

65. The method of claim 61, wherein said monomer comprises polyethylene glycol [PEG] methacrylate and acrylates, poly(acrylic acid), poly(methacrylic acid), 2-diethylaminoethylmethacrylate, 2-aminoethyl methacrylate, poly(ethylene glycol) dimethacrylates and acrylates, acrylamide/bisacrylamide, poly(2-hydroxyethyl methacrylate), methacrylated dextrans, acrylated dextrans, or poly(ethylene glycol)-polyester acrylated/methacrylated block copolymer.

66. The method of claim 62, wherein said block copolymer is acrylated PEG-poly(lactide-co-glycolide) [PLGA]-PEG or PLGA-PEG-PLGA.
